SEQUENCE LISTING (36 sequences)

SEQ ID NO: 1 - protein, 177 aa, Artificial Sequence
  Description of Artificial Sequence artificially synthesized sequence
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - DNA, 462 bp, Artificial Sequence
  Description of Artificial Sequence artificially synthesized sequence
atgttccatgtttcttttagatatatctttggaattcctccactgatccttgttctgctgcctgtcacatcatctgagtgccacattaaagacaaagaaggtaaagcatatgagagtgtactgatgatcagcatcgatgaattggacaaaatgacaggaactgatagtaattgcccgaataatgaaccaaacttttttagaaaacatgtatgtgatgatacaaaggaagctgcttttctaaatcgtgctgctcgcaagttgaagcaatttcttaaaatgaatatcagtgaagaattcaatgtccacttactaacagtatcacaaggcacacaaacactggtgaactgcacaagtaaggaagaaaaaaacgtaaaggaacagaaaaagaatgatgcatgtttcctaaagagactactgagagaaataaaaacttgttggaataaaattttgaagggcagtata

SEQ ID NO: 3 - DNA, 114 bp, Artificial Sequence
  Description of Artificial Sequence artificially synthesized sequence
acattttgacacccccataatatttttccagaattaacagtataaattgcatctcttgttcaagagttccctatcactctctttaatcactactcacagtaacctcaactcctg

SEQ ID NO: 4 - DNA, 881 bp, Artificial Sequence
  Description of Artificial Sequence artificially synthesized sequence
ggaggaaaaactgtttcatacagaaggcgtggaggaaaaactgtttcatacagaaggcgtggaggaaaaactgtttcatacagaaggcgtcaattgtcctcgacggaggaaaaactgtttcatacagaaggcgtggaggaaaaactgtttcatacagaaggcgtggaggaaaaactgtttcatacagaaggcgtcaattgtcccgggacattttgacacccccataatatttttccagaattaacagtataaattgcatctcttgttcaagagttccctatcactctctttaatcactactcacagtaacctcaactcctgcatatgacctagcgctaccggtcgccaccatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacac

SEQ ID NO: 5 - DNA, 57 bp, Artificial Sequence
  Description of Artificial Sequence artificially synthesized sequence
gctactaacttcagcctgctgaagcaggctggagacgtggaggagaaccctggacct

SEQ ID NO: 6 - DNA, 66 bp, Artificial Sequence
  Description of Artificial Sequence artificially synthesized sequence
gtgaaacagactttgaattttgaccttctgaagttggcaggagacgttgagtccaaccctgggccc

SEQ ID NO: 7 - protein, 134 aa, Artificial Sequence
  Description of Artificial Sequence artificially synthesized sequence
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 8 - DNA, 399 bp, Artificial Sequence
  Description of Artificial Sequence artificially synthesized sequence
atggctcagatgatgactctgagcctccttagcctggtcctggctctctgcatcccctggacccaaggcagtgatggagggggtcaggactgctgccttaagtacagccagaagaaaattccctacagtattgtccgaggctataggaagcaagaaccaagtttaggctgtcccatcccggcaatcctgttctcaccccggaagcactctaagcctgagctatgtgcaaaccctgaggaaggctgggtgcagaacctgatgcgccgcctggaccagcctccagccccagggaaacaaagccccggctgcaggaagaaccggggaacctctaagtctggaaagaaaggaaagggctccaagggctgcaagagaactgaacagacacagccctcaagagga

SEQ ID NO: 9 - DNA, 399 bp, Artificial Sequence
  Description of Artificial Sequence artificially synthesized sequence
atggctcagatgatgactctgagcctccttagcctggtcctggctctctgcatcccctggacccaaggcagtgatggagggggacaggactgctgccttaagtacagccagaagaaaattccctacagtattgtccgaggctataggaagcaagaaccaagtttaggctgtcccatcccggcaatcctgttcttaccccggaagcactctaagcctgagctatgtgcaaaccctgaggaaggctgggtgcagaacctgatgcgccgcctggaccagcctccagccccagggaaacaaagccccggctgcaggaagaaccggggaacctctaagtctggaaagaaaggaaagggctccaagggctgcaagagaactgaacagacacagccctcaagagga

SEQ ID NO: 10 - protein, 243 aa, Artificial Sequence
  Description of Artificial Sequence artificially synthesized sequence
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKR

SEQ ID NO: 11 - protein, 98 aa, Artificial Sequence
  Description of Artificial Sequence artificially synthesized sequence
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 12 - DNA, 324 bp, Artificial Sequence
  Description of Artificial Sequence artificially synthesized sequence
atggccccccgtgtgaccccactcctggccttcagcctgctggttctctggaccttcccagccccaactctggggggtgctaatgatgcggaagactgctgcctgtctgtgacccagcgccccatccctgggaacatcgtgaaagccttccgctaccttcttaatgaagatggctgcagggtgcctgctgttgtgttcaccacactaaggggctatcagctctgtgcacctccagaccagccctgggtggatcgcatcatccgaagactgaagaagtcttctgccaagaacaaaggcaacagcaccagaaggagccctgtgtct

SEQ ID NO: 13 - protein, 469 aa, Artificial Sequence
  Description of Artificial Sequence artificially synthesized sequence
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 14 - protein, 467 aa, Artificial Sequence
  Description of Artificial Sequence artificially synthesized sequence
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 15 - protein, 511 aa, Artificial Sequence
  Description of Artificial Sequence artificially synthesized sequence
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 16 - DNA, 194 bp, Artificial Sequence
  Description of Artificial Sequence artificially synthesized sequence
ggaggaaaaactgtttcatacagaaggcgtggaggaaaaactgtttcatacagaaggcgtggaggaaaaactgtttcatacagaaggcgtcaattgtcctcgacggaggaaaaactgtttcatacagaaggcgtggaggaaaaactgtttcatacagaaggcgtggaggaaaaactgtttcatacagaaggcgt

SEQ ID NO: 17 - DNA, 72 bp, Artificial Sequence
  Description of Artificial Sequence artificially synthesized sequence
atctacatctgggcgcccttggccgggacttgtggggtccttctcctgtcactggttatcaccctttactgc

SEQ ID NO: 18 - protein, 42 aa, Artificial Sequence
  Description of Artificial Sequence artificially synthesized sequence
KRGRKKLLYIFKQPFMRPVQTTQEEDGCSCRFPEEEEGGCEL

SEQ ID NO: 19 - DNA, 339 bp, Artificial Sequence
  Description of Artificial Sequence artificially synthesized sequence
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 20 - DNA, 63 bp, Artificial Sequence
  Description of Artificial Sequence artificially synthesized sequence
atggccttaccagtgaccgccttgctcctgccgctggccttgctgctccacgccgccaggccg

SEQ ID NO: 21 - DNA, 135 bp, Artificial Sequence
  Description of Artificial Sequence artificially synthesized sequence
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 22 - DNA, 30 bp, Artificial Sequence
  Description of Artificial Sequence artificially synthesized sequence
ggaggaaaaactgtttcatacagaaggcgt

SEQ ID NO: 23 - protein, 247 aa, Artificial Sequence
  Description of Artificial Sequence artificially synthesized sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 24 - protein, 473 aa, Artificial Sequence
  Description of Artificial Sequence artificially synthesized sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 25 - protein, 471 aa, Artificial Sequence
  Description of Artificial Sequence artificially synthesized sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 26 - protein, 515 aa, Artificial Sequence
  Description of Artificial Sequence artificially synthesized sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 27 - DNA, 729 bp, Artificial Sequence
  Description of Artificial Sequence artificially synthesized sequence
gaggtgcagctggtgcagagcggcgccgaggtgaagaagcccggcgccagcgtgaaggtgagctgcaaggccagcggctacaccttcagcgactacgagatgcactgggtgcggcaggcccccggccagggcctggagtggatgggcgccatccaccccggcagcggcgacaccgcctacaaccagcggttcaagggccgggtgaccatcaccgccgacaagagcaccagcaccgcctacatggagctgagcagcctgcggagcgaggacaccgccgtgtactactgcgcccggttctacagctacgcctactggggccagggcaccctggtgaccgtgagcgccggtggaggcggttcaggcggaggtggttctggcggtggcggatcggacatcgtgatgacccagacccccctgagcctgcccgtgacccccggcgagcccgccagcatcagctgccggagcagccagagcctggtgcacagcaacggcaacacctacctgcagtggtacctgcagaagcccggccagagcccccagctgctgatctacaaggtgagcaaccggttcagcggcgtgcccgaccggttcagcggcagcggcagcggcaccgacttcaccctgaagatcagccgggtggaggccgaggacgtgggcgtgtactactgcagccagagcatctacgtgccctacaccttcggccagggcaccaagctggagatcaaacgt

SEQ ID NO: 28 - DNA, 531 bp, Artificial Sequence
  Description of Artificial Sequence artificially synthesized sequence
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacac

SEQ ID NO: 29 - DNA, 402 bp, Artificial Sequence
  Description of Artificial Sequence artificially synthesized sequence
atggctcagtcactggctctgagcctccttatcctggttctggcctttggcatccccaggacccaaggcagtgatggaggggctcaggactgttgcctcaagtacagccaaaggaagattcccgccaaggttgtccgcagctaccggaagcaggaaccaagcttaggctgctccatcccagctatcctgttcttgccccgcaagcgctctcaggcagagctatgtgcagacccaaaggagctctgggtgcagcagctgatgcagcatctggacaagacaccatccccacagaaaccagcccagggctgcaggaaggacaggggggcctccaagactggcaagaaaggaaagggctccaaaggctgcaagaggactgagcggtcacagacccctaaagggcca

SEQ ID NO: 30 - DNA, 294 bp, Artificial Sequence
  Description of Artificial Sequence artificially synthesized sequence
atggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagt

SEQ ID NO: 31 - protein, 154 aa, Artificial Sequence
  Description of Artificial Sequence artificially synthesized sequence
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 32 - protein, 133 aa, Artificial Sequence
  Description of Artificial Sequence artificially synthesized sequence
MAQMMTLSLLSLVLALCIPWTQGSDGGGQDCCLKYSQKKIPYSIVRGYRKQEPSLGCPIPAILFSPRKHSKPELCANPEEGWVQNLMRRLDQPPAPGKQSPGCRKNRGTSKSGKKGKGSKGCKRTEQTQPSRG

SEQ ID NO: 33 - protein, 133 aa, Artificial Sequence
  Description of Artificial Sequence artificially synthesized sequence
MAQMMTLSLLSLVLALCIPWTQGSDGGGQDCCLKYSQKKIPYSIVRGYRKQEPSLGCPIPAILFLPRKHSKPELCANPEEGWVQNLMRRLDQPPAPGKQSPGCRKNRGTSKSGKKGKGSKGCKRTEQTQPSRG

SEQ ID NO: 34 - protein, 108 aa, Artificial Sequence
  Description of Artificial Sequence artificially synthesized sequence
MAPRVTPLLAFSLLVLWTFPAPTLGGANDAEDCCLSVTQRPIPGNIVKAFRYLLNEDGCRVPAVVFTTLRGYQLCAPPDQPWVDRIIRRLKKSSAKNKGNSTRRSPVS

SEQ ID NO: 35 - protein, 580 aa, Artificial Sequence
  Description of Artificial Sequence artificially synthesized sequence
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 36 - DNA, 126 bp, Artificial Sequence
  Description of Artificial Sequence artificially synthesized sequence
aaacggggcagaaagaaactcctgtatatattcaaacaaccatttatgagaccagtacaaactactcaagaggaagatggctgtagctgccgatttccagaagaagaagaaggaggatgtgaactg

(19) United States
(12) Patent Application Publication
(10) Pub. No.: US 2023/0143947 A1
(43) Pub. Date: May 11, 2023

(54) IMMUNE EFFECTOR CELL IN WHICH EXPRESSION IS REGULATED BY CYTOKINES

(71) Applicant: CRAGE MEDICAL CO., LIMITED, Hong Kong (CN)

(72) Inventors: Zonghai LI, Shanghai (CN); Hua JIANG, Shanghai (CN)

(73) Assignee: CRAGE MEDICAL CO., LIMITED, Hong Kong (CN)

(21) Appl. No.: 17/761,723

(22) PCT Filed: Sep. 21, 2020
(86) PCT No.: PCT/CN2020/116479

§ 371 Date: Mar. 18, 2022

(30) Foreign Application Priority Data
Sep. 20, 2019 (CN) 201910896446.X

(51) Int. Cl. A61K 35/17; C12N 5/0783; C12N 15/86; C07K 14/715; C07K 14/54; C07K 14/555; A61K 45/06; A61P 35/00

(52) U.S. Cl. CPC A61K 35/17; C12N 5/0636; C12N 15/86; C07K 14/7155; C07K 14/5409; C07K 14/54; C07K 14/555; A61K 45/06; A61P 35/00; C12N 2740/15043

(57) ABSTRACT

Provided is a genetically engineered immune effector cell, wherein the immune effector cell expresses a receptor specifically recognizing a target antigen and IL7, and the IL-7 is inducibly expressed and regulated by the receptor. Further provided are a nucleic acid molecule expressing the receptor and IL7 in the immune effector cell and a method for preparing the immune effector cell.

### FIELD OF THE INVENTION

The invention belongs to the field of cell therapy, and relates to genetically engineered cells and applications. More particularly, the present invention relates to the expression of a receptor specifically recognizing a target antigen and IL7, wherein IL-7 is inducibly expressed and regulated by the receptor.v

### BACKGROUND OF THE INVENTION

CAR-T cells may specifically kill tumors in a non-MHC restriction manner, and show good application prospects in tumor immunotherapy, but they still have many limitations, such as poor efficacy on solid tumors, a candidate drug exhibiting excellent effects in vitro often fail to exhibit corresponding effects in vivo.

Some researchers have tried to use cytokine-secreting CAR-T cells for tumor killing, such as co-expression of IL7 on CAR-T cells. However, the applicant of the present application found that expression of cytokines may lead to the death of animals, suggesting that the use of cytokine-secreting CAR-T cells may have safety concerns.

### SUMMARY OF THE INVENTION

The object of the present invention is to provide an immune effector cell that expresses exogenous IL-7 and a receptor specifically recognizing a target antigen, wherein IL-7 is inducibly expressed and regulated by the receptor.

In a first aspect of the present invention, provided is a genetically engineered immune effector cell which expresses a receptor specifically recognizing a target antigen and IL7, wherein the IL-7 is inducibly expressed and regulated by the receptor.

In a preferred embodiment, the expression of IL-7 may be initiated when the receptor recognizes the target antigen.

In a preferred embodiment, the receptor induces the expression of the IL-7 through an inducible promoter.

In a preferred embodiment, the immune effector cell further expresses a chemokine, a chemokine receptor, a cytokine other than IL-7, siRNA reducing PD-1 expression, a protein blocking the binding of PD-L1 to PD-1, or a safety switch.

Preferably, the chemokine is a lymphocyte chemokine; more preferably, the lymphocyte chemokine is CCL21 or CCL19.

Preferably, the chemokine receptor is selected from the group consisting of: CCR2, CCR5, CXCR2, and CXCR4.

Preferably, the other cytokine is selected from the group consisting of: IL-15, IL-21, IL18, and type I interferon.

Preferably, the protein blocking the binding of PD-L1 to PD-1 is selected from the group consisting of: a PD-L1 antibody, a PD-1 antibody, a natural PD-1 or a truncated fragment of the natural PD-1, and a fusion peptide containing the natural PD-1 or the truncated fragment of the natural PD-1.

Preferably, the safety switch is selected from the group consisting of: iCaspase-9, truancated EGFR, RQR8, and a protein with a killing effect on immune effector cells.

In a preferred embodiment, the immune effector cell is selected from the group consisting of: a T cell, an NK cell, an NKT cell, a mast cell, a macrophage, a dendritic cell, a CIK cell, and a stem cell-derived immune effector cell.

In a preferred embodiment, the inducible promoter comprises a binding motif of a transcription factor, and the activation of the inducible promoter is dependent on the activation of the receptor.

In a preferred embodiment, the binding motif comprises an NFAT, NF-κB or AP-1 binding motif, or a combination of at least two of NFAT, NF-KB and AP-1 binding motifs; preferably, the binding motif is an NFAT binding motif.

In a preferred embodiment, the binding motif comprises 1-12 NFAT binding motifs, 1-12 NF-κB binding motifs, 1-12 AP-1 binding motifs, or a combination of at least two of 1-12 NFAT, 1-12 NF-κB and 1-12 AP-1 binding motifs; preferably, the binding motif comprises 1-6 NFAT binding motifs, 1-6 NF-KB binding motifs, 1-6 AP-1 binding motifs, or a combination of at least two of 1-6 NFAT, 1-6 NF-κB and 1-6 AP-1 binding motifs.

In a preferred embodiment, the sequence of the NFAT binding motif is represented by SEQ ID NO: 22.

In a preferred embodiment, the inducible promoter of the immune cell further comprises a minimal promoter operably linked to the binding motif.

In a preferred embodiment, the minimal promoter is a cytokine minimal promoter.

In a preferred embodiment, the minimal promoter includes interleukin, interferon, tumor necrosis factor superfamily, colony stimulating factor, chemokine and growth factor minimal promoter; preferably, the minimal promoter is an IFN-y, a TNF-a, or an IL-2 minimal promoter; more preferably, the minimal promoter is an IL-2 minimal promoter.

In a preferred embodiment, the sequence of the IL-2 minimal promoter is represented by SEQ ID NO: 3.

In a preferred embodiment, the immune effector cell is a T cell.

In a preferred embodiment,

the IL-7 is natural IL-7, or a truncated fragment of natural IL-7 or a mutant of natural IL-7 having the same function as natural IL-7; preferably, the natural IL-7 has at least 90% identity with the amino acid sequence represented by SEQ ID NO: 1 or SEQ ID NO: 31, or is a truncated fragment of the amino acid sequence represented by SEQ ID NO: 1 or SEQ ID NO: 31; alternatively, has at least 90% identity with the amino acid sequence encoded by the nucleotide represented by SEQ ID NO: 2 or SEQ ID NO: 28, or is a truncated fragment of the amino acid sequence encoded by the nucleotide represented by SEQ ID NO: 2 or SEQ ID NO: 28.

In a preferred embodiment, the CCL21 is natural CCL21, or a truncated fragment of natural CCL21 or a mutant of natural CCL21 having the same function as natural CCL21;

preferably, the natural CCL21 has at least 90% identity with the amino acid sequence represented by SEQ ID NO: 7, SEQ ID NO: 32 or SEQ ID NO: 33, or is a truncated fragment of the amino acid sequence represented by SEQ ID NO: 7, SEQ ID NO: 32 or SEQ ID NO: 33; alternatively, has at least 90% identity with the amino acid sequence encoded by the nucleotide represented by SEQ ID NO: 8, SEQ ID NO: 9 or SEQ ID NO: 29, or is a truncated fragment of the amino acid sequence encoded by the nucleotide represented by SEQ ID NO: 8, SEQ ID NO: 9 or SEQ ID NO: 29.

In a preferred embodiment, the CCL19 is natural CCL19, or a truncated fragment of natural CCL19 or a mutant of natural CCL19 having the same function as natural CCL19; preferably, the natural CCL19 has at least 90% identity with the amino acid sequence represented by SEQ ID NO: 11 or SEQ ID NO: 34, or is a truncated fragment of the amino acid sequence represented by SEQ ID NO: 11 or SEQ ID NO: 34; alternatively, has at least 90% identity with the amino acid sequence encoded by the nucleotide represented by SEQ ID NO: 12 or SEQ ID NO: 30, or is a truncated fragment of the amino acid sequence encoded by the nucleotide represented by SEQ ID NO: 12 or SEQ ID NO: 30.

In a preferred embodiment, the CCL19 is natural CCL19, or a truncated fragment of natural CCL19 or a mutant of natural CCL19 having the same function as natural CCL19.

In a preferred embodiment, the chemokine, the chemokine receptor, the cytokine other than IL-7, the siRNA reducing PD-1 expression, the protein blocking the binding of PD-L1 to PD-1, or the safety switch is either constitutively expressed or inducibly expressed.

In a preferred embodiment, the target antigen is a tumor antigen and/or a pathogen antigen; preferably, a tumor antigen.

In a particular embodiment, the target antigen is a tumor antigen, and in a preferred embodiment, the tumor antigen is selected from the group consisting of: thyroid-stimulating hormone receptor (TSHR); CD171; CS-1; C-type lectin-like molecule-1; ganglioside GD3; Tn antigen; CD19; CD20; CD 22; CD 30; CD 70; CD 123; CD 138; CD33; CD44; CD44v7/8; CD38; CD44v6; B7H3(CD276), B7H6; KIT (CD117); interleukin 13 receptor subunit alpha (IL-13Rα); interleukin 11 receptor alpha (IL-11Rα); prostate stem cell antigen (PSCA); prostate specific membrane antigen (PSMA); carcinoembryonic antigen (CEA) ; NY-ESO-1; HIV-1 Gag; MART-1; gp100; tyrosinase; mesothelin; EpCAM; protease serine 21 (PRSS21); vascular endothelial growth factor receptor; Lewis (Y) antigen; CD24; platelet-derived growth factor receptor beta (PDGFR-β); stage-specific embryonic antigen-4 (SSEA-4); cell surface associated mucin 1 (MUC1), MUC6; epidermal growth factor 20 receptor family and its mutants (EGFR, EGFR2, ERBB3, ERBB4, EGFRvIII); neural cell adhesion molecule (NCAM); carbonic anhydrase IX (CAIX); LMP2; ephrin A receptor 2 (EphA2); fucosyl GM1; sialyl Lewis adhesion molecule (sLe); ganglioside GM3; TGS5; high molecular weight melanoma-associated antigen (HMWMAA); ortho-acetyl GD2 ganglioside (OAcGD2); folate receptor; tumor endothelial marker 25 1 (TEM1/CD248); tumor endothelial marker 7-related (TEM7R); Claudin 6, Claudin18.2 (CLD18A2), Claudin18.1; ASGPR1; CDH16; 5T4; 8H9; αvβ6 integrin; B cell maturation antigen (BCMA); CA9; kappa light chain; CSPG4; EGP2, EGP40; FAP; FAR; FBP; embryonic AchR; HLA-A1, HLA-A2; MAGEA1, MAGE3; KDR; MCSP; NKG2D ligand; PSC1; ROR1; Sp17; SURVIVIN; TAG72; TEM1; fibronectin; tenascin; carcinoembryonic variant of tumor necrosis region; G protein-coupled receptor class C group 5-member D (GPRCSD); X chromosome open reading frame 61 (CXORF61); CD97; CD179a; anaplastic lymphoma kinase (ALK); polysialic acid; placenta specific 1 (PLAC1); hexose moiety of globoH glycoceramide (GloboH); mammary gland differentiation antigen (NY-BR-1); uroplakin 2 (UPK2); hepatitis A virus cell receptor 1 (HAVCR1); adrenergic receptor 5 beta 3 (ADRB3); pannexin 3 (PANX3); G protein-coupled receptor 20 (GPR20); lymphocyte antigen 6 complex locus K9 (LY6K); olfactory receptor 51E2 (OR51E2); TCRγ alternate reading frame protein (TARP); Wilms tumor protein (WTI); ETS translocation variant 6 (ETV6-AML); sperm protein 17 (SPA17); X antigen family member 1A (XAGE1); angiopoietin-binding cell surface receptor 2 (Tie2); melanoma cancer testis antigen-1 (MAD-CT-1); melanoma cancer testis antigen-2 (MAD-CT-2); Fos-associated antigen 1; p53 mutant; human telomerase reverse transcriptase (hTERT); sarcoma translocation breakpoint; melanoma inhibitor of apoptosis (ML-IAP); ERG (transmembrane protease serine 2 (TMPRSS2) ETS fusion gene); N-acetylglucosaminyltransferase V (NA17); paired box protein Pax-3 (PAX3); androgen receptor; cyclin B1; V-myc avian myelocytomatosis virus oncogene neuroblastoma-derived homolog (MYCN); Ras homolog family member C (RhoC); cytochrome P450 1B1 (CYP1B1); CCCTC-binding factor (zinc finger protein)-like (BORIS); squamous cell carcinoma antigen 3 (SART3) recognized by T cells; paired box protein Pax-5 (PAXS); proacrosin binding protein sp32 (OYTES1); lymphocyte-specific protein tyrosine kinase (LCK); A-kinase-anchored protein 4 (AKAP-4); synovial sarcoma X breakpoint 2 (SSX2); CD79a; CD79b; CD72; leukocytes related immunoglobulin-like receptor 1 (LAIR1); Fc fragment of IgA receptor (FCAR); leukocyte immunoglobulin-like receptor subfamily member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); immunoglobulin lambda-like polypeptide 1 (IGLL1).

In particular embodiments, the target antigen is a pathogen antigen. In a preferred embodiment, the pathogen antigen is selected from: antigens from viruses, bacteria, fungi, protozoa, or parasites. In a particular embodiment, the viral antigen is selected from the group consisting of: cytomegalovirus antigen, Epstein-Barr virus antigen, human immunodeficiency virus antigen, and influenza virus antigen.

In a preferred embodiment, the target antigen is a solid tumor antigen; preferably, the solid tumor antigen is GPC3, EGFR, EGFRvIII, mesothelin, or Claudin18.2. In a preferred embodiment, the solid tumor antigen is GPC3.

In a preferred embodiment, the receptor is selected from the group consisting of: a chimeric antigen receptor (CAR), a T cell receptor (TCR), a T cell fusion protein (TFP), a T cell antigen coupler (TAC), and a combination thereof; preferably, the receptor is a chimeric antigen receptor.

In a preferred embodiment, the chimeric antigen receptor comprises:

(i) an antibody or fragment thereof specifically binding the target antigen, a transmembrane domain of CD28 or CD8, a costimulatory signaling domain of CD28, and an intracellular signaling domain of CD3ζ; or

(ii) an antibody or fragment thereof specifically binding the target antigen, a transmembrane domain of CD28 or CD8, a costimulatory signaling domain of 4-1BB, and an intracellular signaling domain of CD3ζ; or

(iii) an antibody or fragment thereof specifically binding the target antigen, a transmembrane domain of CD28 or CD8, a costimulatory signaling domain of CD28, a costimulatory signaling domain of 4-1BB, and an intracellular signaling domain of CD3ζ.

In a preferred embodiment, the amino acid sequence of the antigen binding domain of the chimeric antigen receptor (CAR), T cell fusion protein (TFP), or T cell antigen coupler (TAC) has at least 90% identity with the amino acid sequence represented by SEQ ID NO: 10 or SEQ ID NO: 23.

In a preferred embodiment, the amino acid sequence of the receptor has at least 90% identity with the amino acid sequence represented by SEQ ID NO: 13, 14, 15, 24, 25, or 26.

In a preferred embodiment, the nucleic acid sequence of the immune cell-inducible promoter inducing the expression of IL7 is represented by SEQ ID NO: 4.

In a preferred embodiment, the receptor and IL-7 in the genetically engineered immune effector cells of the present invention are expressed by the same nucleic acid molecule, or expressed by different nucleic acid molecules.

In a preferred embodiment, the receptor and IL-7 are expressed by the same nucleic acid molecule; an expression cassette of the IL-7 and the receptor, and an expression cassette and another expression cassette are directly connected or connected by a tandem fragment, wherein the tandem fragment is selected from the group consisting of: F2A, PA2, T2A, and/or E2A.

In a second aspect of the present invention, provided is a nucleic acid molecule expressing the above-mentioned IL-7 of the present invention, or IL-7 and a chemokine, a chemokine receptor, a cytokine other than IL-7, a siRNA reducing PD-1 expression, a protein blocking the binding of PD-L1 to PD-1, or a safety switch; and the nucleic acid molecule also expresses the above-mentioned receptor of the present invention that specifically recognizes the target antigen;

In a preferred embodiment, the nucleic acid consists of DNA and/or RNA;

In a preferred embodiment, the nucleic acid is mRNA;

In a preferred embodiment, the nucleic acid comprises a nucleotide analog.

In a third aspect of the present invention, provided is a vector comprising the above-mentioned nucleic acid molecule of the present invention;

In a preferred embodiment, the vector is selected from the group consisting of: DNA, RNA, a plasmid, a lentiviral vector, an adenoviral vector, an Rous sarcoma virus (RSV) vector, and a reverse transcription viral vector.

In a fourth aspect of the present invention, provided is a cell which comprises the above-mentioned vector of the present invention or a cell whose genome is integrated with the above-mentioned nucleic acid molecule of the present invention;

In a preferred embodiment, the cells is a human T cell, preferably an allogeneic T cell.

In a fifth aspect of the present invention, provided is a method for preparing cells, comprising transducing T cells with the vector of the present invention or the nucleic acid molecule of the present invention.

In a sixth aspect of the present invention, provided is a method for producing an RNA-engineered cell population, comprising introducing an in vitro transcribed RNA or a synthetic RNA into a cell, wherein the RNA comprises the above-mentioned nucleic acid of the present invention.

In a sixth aspect of the present invention, provided is a method for providing anti-tumor immunity in a mammal, comprising administering an effective amount of the above-mentioned cells of the present invention, the nucleic acid molecules of the present invention, and the vectors of the present invention to the mammal;

In a preferred embodiment, the mammal is a human.

In a seventh aspect of the present invention, provided is a method for treating a mammal suffering from a disease related to the expression of GPC3 or claudin18.2, comprising administering an effective amount of the cells of the present invention, the nucleic acid molecules of the present invention, or the vectors of the present invention to the mammal;

In a preferred embodiment, the disease related to the expression of GPC3 or claudin18.2 is selected from the group consisting of: colon cancer, rectal cancer, renal cell cancer, liver cancer, lung cancer, small intestine cancer, esophagus cancer, melanoma, bone cancer, pancreatic cancer, skin cancer, head and neck cancer, skin or intraocular malignant melanoma, uterine cancer, ovarian cancer, rectal cancer, anal cancer, stomach cancer, testicular cancer, uterine cancer, fallopian tube cancer, endometrial cancer, cervical cancer, vaginal cancer, endocrine system cancer, thyroid cancer, parathyroid cancer, adrenal cancer, soft tissue sarcoma, urethral cancer, penile cancer, bladder cancer, kidney or ureter cancer, renal pelvis cancer, central nervous system (CNS) tumor, tumor angiogenesis, spinal tumor, brain stem glial tumor, pituitary adenoma, Kaposi's sarcoma, epidermoid carcinoma, squamous cell carcinoma, non-cancer related indications related to GPC3 or claudin18.2 expression; preferably, selected from the group consisting of: liver cancer, lung cancer, breast cancer, ovarian cancer, kidney cancer, thyroid cancer, stomach cancer, colorectal cancer, pancreatic cancer, and esophageal cancer.

In a preferred embodiment, the cells according to any one of claims 1-31 and 36 are administered in combination with an agent increasing the efficacy of the cells of any one of claims 1-31 and 36; preferably, in combination with a chemotherapeutic agent.

In a preferred embodiment, the cells according to any one of claims 1-31 and 36 are administered in combination with an agent ameliorating one or more side effects associated with the administration of the cells according to any one of claims 1-31 and 36;

In a preferred embodiment, the cells according to any one of claims 1-31 and 36 are administered in combination with an agent for treating a disease associated with GPC3 or claudin18.2, preferably in combination with a chemotherapeutic agent.

In a eighth aspect of the present invention, provided is use of the cell of the present invention, the nucleic acid molecule of the present invention, and the vector of the present invention for preparaing a medicament, preferably for preparaing a medicament for inhibiting tumors or inhibiting pathogens. In a preferred embodiment, the use is for preparing a medicament for inhibiting tumors or inhibiting pathogens.

In a ninth aspect of the present invention, provided is a pharmaceutical composition which comprises the cells of the present invention and a pharmaceutically acceptable carrier or excipient.

Beneficial Effects of the Present Invention

1. The immune effector cells provided by the present invention improve the application safety of CAR-T cells by regulating the expression and secretion of IL-7.

2. The applicant also found that the immune effector cells in which the expression of IL-7 is regulated may also enhance the anti-tumor effect of CAR-T.

### BRIEF DESCRIPTION OF THE DRAWINGS

FIG. 1 shows plasmid diagrams of PRRLSIN-GPC3-BBZ-NFAT-IL7, PRRLSIN-GPC3-BBZ-CCL21-NFAT-IL7, PRRLSIN-GPC3-BBZ-CCL21-IL7, PRRLSIN-GPC3-BBZ-CCL19-NFAT-IL7, PRRLSIN-GPC3-BBZ-CCL19-IL7, and PRRLSIN-GPC3-BBZ-IL7;

FIG. 2 shows the results of the positive rate of NFAT-7*21-CAR-T cells and 7*21-CAR-T cells;

FIG. 3 shows the results of the positive rate of NFAT-7*19-CAR-T cells and 7*19-CAR-T cells;

FIG. 4A shows the killing of tumor cells by NFAT-7*21-CAR-T cells and 7*21-CAR-T cells;

FIG. 4B shows the killing of tumor cells by NFAT-7*19-CAR-T cells and 7*19-CAR-T cells;

FIG. 5 shows the cytokine secretion of NFAT-7*21-CAR-T cells and 7*21-CAR-T cells;

FIG. 6 shows the cytokine secretion of NFAT-7*19-CAR-T cells and 7*19-CAR-T cells;

FIG. 7A shows the in vivo tumor inhibition results of NFAT-7*21-CAR-T cells and

7*21-CAR-T cells; FIG. 7B shows the effect of NFAT-7*21-CAR-T cells and 7*21-CAR-T cells on the body weight of mice;

FIG. 8A shows the in vivo tumor inhibition results of NFAT-7*19-CAR-T cells and 7*19-CAR-T cells; FIG. 8B shows the effect of NFAT-7*19-CAR-T cells and 7*19-CAR-T cells on the body weight of mice;

FIG. 9 shows the tumor suppression results of NFAT-IL7-CAR-T cells and IL7-CAR-T cells.

### DETAIL DESCRIPTION OF THE INVENTION

The present invention finds that for CAR-T cells co-expressing IL-7, regulating the expression of IL-7 may not only improve the safety of CAR-T cells, but also exhibit more excellent tumor killing effect; even for refractory solid tumors, the CAR-T cells also show more excellent anti-tumor ability.

Based on the present disclosure, the ordinary skilled person in the art should appreciate that many modification or changes may be made in the disclosed particular embodiments to obtain a like or similar result without departing from the spirit and scope described herein. The scope of the present invention is not to be limited by the particular embodiments described herein, which are merely intended to illustrate various aspects described herein, and functionally equivalent methods and components are considered to be included within the scope of the present application.

Unless specifically defined, all technical and scientific terms used herein have the meanings commonly understood by those skilled in the fields of gene therapy, biochemistry, genetics and molecular biology. All methods and materials similar or equivalent to those described herein may be used in the practice or testing described herein. These techniques such as methods and materials are well documented in the literatures, see for example, unless otherwise stated, the practice of the present invention will employ conventional techniques of cell biology, cell culture, molecular biology, transgenic biology, microbiology, recombinant DNA, and immunology, they all belong to the technical scope of the art, Current Protocols in Molecular Biology (Frederick M. Ausubel, 2000, Wileyand sonInc, Library of Congress, USA); Molecular Cloning: A Laboratory Manual, Third Edition, (Sambrooketal, 2001, Cold Spring Harbor, N.Y.: Cold Spring Harbor Laboratory Press); Oligonucleotide Synthesis (M.J.Gaited., 1984); Mullis et al. U.S. Pat. No. 4,683,195; Nucleic Acid Hybridization (B. D. Harries & S. J. Higginseds. 1984); Transcription and Translation (B. D. Hames & S. J. Higginseds. 1984); Culture of Animal Cells (R. I. Freshney, Alan R. Liss, Inc., 1987); Immobilized Cells and Enzymes (IRL Press, 1986); B. Perbal, A Practical Guide to Molecular Cloning (1984); the series, Methods in Enzymology (J. Abelson and M. Simon, eds.-in-chief, Academic Press, Inc., New York), especially Vols. 154 and 155 (Wuetal. eds.) and Vol.185, “Gene Expression Technology” (D. Goeddel, ed.); Gene Transfer Vectors for Mammalian Cells ((J. H. Miller and M P Caloseds, 1987, Cold Spring Harbor Laboratory); Immunochemical Methods in Cell and Molecular Biology (Mayer and Walker, eds., Academic Press, London, 1987); Hand Book of Experimental Immunology, Volumes I-IV (D. M. Weir and C. C. Blackwell, eds., 1986); and Manipulating the Mouse Embryo (Cold Spring Harbor Laboratory Press, Cold Spring Harbor, N.Y., 1986).

All publications, patent applications, patents, and other references mentioned herein are incorporated by reference in their entirety. In case of conflict, the present specification will control. Furthermore, unless otherwise specified, the materials, methods, and examples set forth in this specification are illustrative only and not intended to be limiting.

The term “engineered” as used herein may refer to one or more alterations of a nucleic acid, e.g., a nucleic acid within the genome of an organism. The term “engineered” may refer to a change, addition and/or deletion of a gene. An engineered cell may also refer to a cell with an added, deleted and/or altered gene.

The term “genetically engineered cell” as used herein refers to a cell engineered by means of genetic engineering. In some embodiments, the cell is an immune effector cell. In some embodiments, the cell is a T cell. In some embodiments, the genetically engineered cell described herein refers to a cell expressing an exogenous receptor that specifically binds a target antigen. In some embodiments, the genetically engineered cell described herein refers to an immune effector cell that expresses an exogenous receptor specifically binding a target antigen, and induces the expression of exogenous IL7. In some embodiments, the genetically engineered cell described herein refers to an immune effector cell that expresses an exogenous receptor specifically binding to a target antigen, expresses exogenous CLL21, and induces the expression of exogenous IL7. In some embodiments, the genetically engineered cell described herein refers to an immune effector cell that expresses an exogenous receptor specifically binding to a target antigen, expresses exogenous CLL19, and induces the expression of exogenous IL7. In some embodiments, the genetically engineered cell described herein may also be a T cell that co-expresses a chimeric antigen receptor specifically binding a tumor antigen, induces the expression of exogenous IL7 and a protein promoting T cell proliferation. In some embodiments, the genetically engineered cell described herein may also be a T cell that co-expresses a chimeric antigen receptor specifically binding a tumor antigen, expresses exogenous CLL21, and induces the expression of exogenous IL7. In some embodiments, the genetically engineered cell described herein may also be a T cell that co-expresses a chimeric antigen receptor specifically binding a tumor antigen, expresses exogenous CLL19, and induces the expression of exogenous IL7. In some embodiments, the genetically engineered cell described herein may also be a T cell that co-expresses a chimeric antigen receptor specifically binding a tumor antigen, CLL21, and induces the expression of an IL-7R binding protein or exogenous IL-7. In some embodiments, the genetically engineered cell described herein may also be a T cell that co-expresses a chimeric antigen receptor specifically binding a tumor antigen, CLL19, and induces the expression of an IL-7R binding protein or exogenous IL-7.

The term “immune effector cell” refers to a cell involved in an immune response producing immune effectors, such as a T cell, a B cell, a natural killer (NK) cell, a natural killer T (NKT) cell, a mast cell and a bone marrow-derived phagocyte, a macrophage, a dendritic cell, a CIK cell, or a stem cell-derived immune effector cell. In some embodiments, the immune effector cell is a T cell, NK cell, NKT cell. In some embodiments, the T cell may be an autologous T cell, xenogeneic T cell, allogeneic T cell. In some embodiments, the NK cell may be an allogeneic NK cell.

The terms “peptide”, “polypeptide” and “protein” are used interchangeably, and refer to a compound consisting of amino acid residues covalently linked by peptide bonds. A protein or peptide must contain at least two amino acids, and there is no limit to the maximum number of amino acids that may include the sequence of a protein or peptide. Polypeptides include any peptide or protein having two or more amino acids bound to each other by peptide bonds. As used herein, the term refers to short chains (also commonly referred to in the art as e.g., peptides, oligopeptides and oligomers), as well as longer chains (also commonly referred to in the art as proteins, which exist in many types). “Polypeptide” includes, for example, biologically active fragments, substantially homologous polypeptides, oligopeptides, homodimers, heterodimers, variants of polypeptides, modified polypeptides, derivatives, analogs, fusion proteins, and the like. Polypeptides include natural peptides, recombinant peptides, or combinations thereof.

The term “IL-7 (interleukin7)” has one of the following characteristics: (i) an amino acid sequence of naturally occurring mammalian IL-7 or a fragment thereof, e.g., the amino acid sequence represented by SEQ ID NO: 1 (human) or SEQ ID NO: 31 (murine), or a fragment thereof; (ii) an amino acid sequence substantially has, for example, at least 85%, 90%, 95%, 96%, 97%, 98%, 99% homology with the amino acid sequence represented by SEQ ID NO: 1 (human) or SEQ ID NO: 31 (murine), or a fragment thereof; (iii) an amino acid sequence encoded by a naturally occurring mammalian IL-7 nucleotide sequence or a fragment thereof, e.g., SEQ ID NO: 28 (human) or a fragment thereof, or SEQ ID NO: 2 (murine) or a fragment thereof; (iv) an amino acid sequence encoded by the nucleotide sequence having, for example, at least 85%, 90%, 95%, 96%, 97%, 98%, 99% homology with the nucleotide sequence represented by SEQ ID NO: 28 (human) or a fragment thereof, or SEQ ID NO: 2 (murine) or a fragment thereof; (v) an amino acid sequence encoded by a degenerate nucleotide sequence of a naturally occurring IL-7 nucleotide sequence or a fragment thereof (e.g., SEQ ID NO: 28 (human) or a fragment thereof, or SEQ ID NO: 2 (murine) or a fragment thereof); or (vi) a nucleotide sequence that hybridizes to one of the foregoing nucleotide sequences under a stringent condition.

IL-7 may interact with (e.g., bind to) IL-7R (preferably IL-7R from mammals, e.g., murine or human), preferably IL-7R from mammals, e.g., murine or human. IL-7 may also exert antitumor effects through non-IL-7R pathways.

“Exogenous IL-7R binding protein” refers to all proteins that may specifically bind to IL-7R and enhance the activity of IL-7R. “Enhancing IL-7R activity” is understood to mean that an IL-7R binding protein is capable of enhancing any one or more activities of naturally occurring IL-7R, including but not limited to: stimulating NK cell proliferation, cytotoxicity or maturation; stimulating proliferation or differentiation of B cells and T cells; stimulating antibody production and affinity maturation in B cells; stimulating cytotoxicity of CD8+T cells; stimulating interferon gamma production in T cells and NK cells; inhibiting activation and maturation of dendritic cells (DC); inhibiting the release of inflammatory mediators from mast cells; enhancing phagocytosis of macrophages; inhibiting TReg cell production or survival; and stimulating proliferation of myeloid progenitor cells.

“CCL21 (Chemokine (C-C motif) ligand 21)” is one of the major immunochemokines, which is expressed in the T cell region of secondary lymphoid tissues of the spleen and lymph nodes, and responsible for recruitment of antigen-activated (mature) dendritic cells (DCs), immature DCs and naive T cells. In the present invention, CCL21 has one of the following features: (i) an amino acid sequence of naturally occurring mammalian CCL21 or a fragment thereof, e.g., the amino acid sequence represented by SEQ ID NO: 7 (human), or SEQ ID NO: 32, 33 (murine), or a fragment thereof; (ii) an amino acid sequence having, for example, at least 85%, 90%, 95%, 96%, 97%, 98%, 99% homology with the amino acid sequence represented by SEQ ID NO: 7 (human), or SEQ ID NO: 32, 33 (murine), or a fragment thereof; (iii) an amino acid sequence encoded by a naturally occurring mammalian CCL21 nucleotide sequence or a fragment thereof (such as SEQ ID NO: 29 (human) or a fragment thereof, or SEQ ID NO: 8, 9 (murine) or a fragment thereof); (iv) an amino acid sequence encoded by a nucleotide sequence having, for example, at least 85%, 90%, 95%, 96%, 97%, 98%, 99% homology with the nucleotide sequence represented by SEQ ID NO: 29 (human), or SEQ ID NO: 8, 9 (murine), or a fragment thereof; (v) an amino acid sequence encoded by a degenerate nucleotide sequence of a naturally occurring CCL21 nucleotide sequence or a fragment thereof (e.g., SEQ ID NO: 29 (human) or a fragment thereof, SEQ ID NO: 8 or 9 (murine) or a fragment thereof); or (vi) a nucleotide sequence that hybridizes to one of the foregoing nucleotide sequences under a stringent condition.

“CCL19 (Chemokine (C-C motif) ligand 19)” is one of the major immunochemokines, which is expressed in the T cell region of secondary lymphoid tissue in the spleen and lymph nodes, and responsible for recruitment of antigen-activated (mature) dendritic cells (DCs), immature DCs and naive T cells. CCL19 in the present invention has one of the following features: (i) an amino acid sequence of naturally occurring mammalian CCL19 or a fragment thereof, e.g., the amino acid sequence represented by SEQ ID NO: 11 (human), or SEQ ID NO: 34 (murine), or a fragment thereof; (ii) an amino acid sequence having, for example, at least 85%, 90%, 95%, 96%, 97%, 98%, 99% homology with the amino acid sequence represented by SEQ ID NO: 11 (human), or SEQ ID NO: 34 (murine), or a fragment thereof; (iii) an amino acid sequence encoded by a naturally occurring mammalian CCL19 nucleotide sequence or a fragment thereof (e.g., SEQ ID NO: 30 (human) or a fragment thereof, or SEQ ID NO: 12 (murine) or a fragment thereof); (iv) an amino acid sequence encoded by a nucleotide sequence having, for example, at least 85% 90%, 95%, 96%, 97%, 98%, 99% homology with the nucleotide sequence represented by SEQ ID NO: 30 (human), or SEQ ID NO: 12 (murine), or a fragment thereof; (v) an amino acid sequence encoded by a degenerate nucleotide sequence of a naturally occurring CCL19 nucleotide sequence or a fragment thereof e.g., SEQ ID NO: 30 (human) or a fragment thereof, or SEQ ID NO: 12 (murine) or a fragment thereof); or (vi) a nucleotide sequence that hybridizes to one of the foregoing nucleotide sequences under a stringent condition.

The term “amino acid modification” includes substitution, addition and/or deletion of an amino acid, “amino acid substitution” means replacing an amino acid at a particular position in the parent polypeptide sequence with another amino acid. As used herein, “amino acid insertion” means the addition of an amino acid at a particular position in the parent polypeptide sequence. As used herein, “amino acid deletion” or “deletion” means the removal of an amino acid at a particular position in the parent polypeptide sequence. The term “conservative modification” as used herein means an amino acid modification that does not significantly affect or alter the binding characteristics of an antibody comprising the amino acid sequence. Such conservative modifications include substitution, insertion and deletion of an amino acid. A modification may be introduced into an antibody of the invention by standard techniques known in the art, such as site-directed mutagenesis, and PCR-mediated mutagenesis. A conservative amino acid substitution is such a substitution in which an amino acid residue is replaced with another amino acid residue having similar side chain. Families of amino acid residues with similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, serine, threonine, tyrosine, cysteine, tryptophan), non-polar side chains (e.g., alanine, valine, leucine, isoleucine, proline, phenylalanine, methionine), beta branched side chains (e.g., threonine, valine, isoleucine), and aromatic side chains (e.g., tyrosine, phenylalanine, tryptophan, histidine).

As used herein, the terms “wild-type”, “ parent” and “natural” represent the same meaning when referring to a protein and DNA. The term “mutation”, “variant” or “mutant” has the same or better biological activity than the natural protein or natural DNA, and has one or more amino acid substitutions, additions or deletions in the amino acid sequence of the natural protein; or has one or more nucleotide substitutions, additions or deletions in the nucleic acid sequence of natural DNA. In particular embodiments, a mutant herein has a sequence having at least about 80%, preferably at least about 90%, more preferably at least about 95%, more preferably at least about 97%, more preferably at least about 98%, most preferably at least about 99% identity with the amino acid sequence of a natural protein or with the nucleic acid sequence of natural DNA. For example, “variant of IL-7” generally refers to a polypeptide obtained by amino acid modification of wild-type IL-7 and having similar biological activity or better biological activity as compared with the original IL-7. The term “truncated fragment” refers to a non-full-length form of a natural protein or natural DNA having contiguous or noncontiguous deletions of multiple amino acid residues or nucleotides in the natural amino acid sequence or nucleic acid sequence, the deletions occur anywhere in the sequence, such as head, middle, tail, and a combination thereof. In the present invention, the truncated fragment of the protein still retains the same function as the natural protein from which it is derived.

“Constitutive expression” also known as continuous expression, refers to the continuous expression of a gene in a cell under almost all physiological conditions. “Inducible expression” refers to expression under a certain condition, e.g., when a T cell bind to an antigen.

The term “effective amount” refers to an amount of a compound, formulation, substance or composition effective to achieve a specific biological result, for example, including but not limited to, an amount or dose sufficient to promote a T cell response. When referring to “immunologically effective amount”, “anti-tumor effective amount”, “tumor-inhibitory effective amount” or “therapeutically effective amount”, the precise dose of the immune effector cells, or therapeutic agent of the present invention, may be determined by a physician taking account of the individual's age, weight, tumor size, or extent of metastasis, and the condition of the patient (subject). An effective amount of immune effector cells refers to, including but not limited to, the number of immune effector cells that may increase, enhance or prolong the antitumor activity of the immune effector cells; including but not limited to, the number of immune effector cells that may increase the number of antitumor immune effector cells or activated immune effector cells; including but not limited to, the number of immune effector cells that may promote IFN-y secretion, tumor regression, tumor shrinkage, and tumor necrosis.

In some embodiments, the antigen-binding receptors described herein refer to chimeric receptors. As used herein “chimeric receptor” refers to a fusion molecule formed by linking DNA fragments or protein-corresponding cDNAs from different sources through gene recombination technology. A chimeric receptor typically includes an extracellular domain, a transmembrane domain, and an intracellular domain. Chimeric receptors that may be used in the present invention include, but are not limited to: chimeric antigen receptor (CAR), modified T cell (antigen) receptor (TCR), T cell fusion protein (TFP), T cell antigen coupler (TAC).

The term “open reading frame (ORF)” is the normal nucleotide sequence of a structural gene, the reading frame from the start codon to the stop codon may encodes a complete polypeptide chain, and there is no stop codon interrupting translation within the ORF.

As used herein “chimeric antigen receptor” or “CAR” refers to a group of polypeptides, when existing in an immune effector cell, they provide said cell with specific recognition for a target cell (usually a cancer cell or tumor cell), and have intracellular signal generation. CARs typically include at least one extracellular antigen-binding domain (also known as an extracellular region, or extracellular antigen-binding region, or an antibody or a fragment thereof specifically binding a target antigen), a transmembrane domain (also known as a transmembrane region), and a cytoplasmic signaling domain (also referred to herein as an “intracellular signaling domain” or “intracellular region”), comprising a functional signaling domain derived from stimulatory and/or costimulatory molecules as defined below. In certain aspects, a group of polypeptides are contiguous with each other. The group of polypeptides includes a dimerization switch that may couple polypeptides to each other in the presence of a dimerization molecule, e.g., an antigen binding domain may be coupled to an intracellular signaling domain. In one aspect, the stimulatory molecule is a delta (ζ) chain bound to a T cell receptor complex. In one aspect, the cytoplasmic signaling domain further comprises one or more functional signaling domains derived from at least one costimulatory molecule as defined below. In one aspect, the costimulatory molecule is selected from the group consisting of: costimulatory molecules described herein, such as 4-1BB (i.e., CD137), CD27, CD28, or a combination thereof. In one aspect, the CAR comprises a chimeric fusion protein, comprising: an extracellular antigen binding domain, a transmembrane domain, and an intracellular signaling domain comprising a functional signaling domain derived from a stimulatory molecule. In one aspect, the CAR comprises a chimeric fusion protein, comprising: an extracellular antigen binding domain, a transmembrane domain, and an intracellular signaling domain comprising a functional signaling domain derived from a costimulatory molecule and a functional signaling domain derived from a stimulatory molecule. In one aspect, the CAR comprises a chimeric fusion protein, comprising: an extracellular antigen binding domain, a transmembrane domain, and an intracellular signaling domain comprising two functional signaling domains derived from one or more costimulatory molecules.

In one aspect, the invention contemplates modification of the amino acid sequence of the original antibody or a fragment thereof (e.g., scFv) that produces a functionally equivalent molecule. For example, the VH or VL of an antigen binding domain of a cancer or tumor associated antigen described herein, such as an scFv contained in a CAR, may be modified to retain at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity with the original VH or VL framework region (e.g., scFv) of an antigen binding domain of a cancer or tumor associated antigen described herein. The present invention contemplates modification of the entire CAR construct, such as modification of one or more amino acid sequences of multiple domains of the CAR construct, to produce a functionally equivalent molecule. The CAR construct may be modified to retain at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity with the original CAR construct.

As used herein, a “transmembrane domain” (also referred to as a transmembrane region) may comprise one or more amino acid fragments adjacent to the transmembrane region, e.g., one or more amino acids associated with the extracellular region of the protein from which the transmembrane protein is derived (e.g., 1, 2, 3, 4, 5, 6, 7, 8, 9, 10, up to 15 amino acids of the extracellular region). In one aspect, the transmembrane domain is a domain associated with one of the other domains of the chimeric receptor, e.g., in one embodiment, the transmembrane domain may be from the same protein from which the signaling domain, costimulatory domain, or hinge domain is derived. In certain instances, a transmembrane domain may be selected or substituted by amino acids to prevent such a domain from binding to a transmembrane domain of the same or different surface membrane protein, for example, which minimize the interaction with other member of the receptor complex comprising the transmembrane domain. In one aspect, the transmembrane domain is capable of homodimerizing with another chimeric receptor on the cell surface of the cell expressing the chimeric receptor. In one aspect, the amino acid sequence of the transmembrane domain may be modified or substituted, so as to minimize interaction with the binding domain of the natural binding partner present in a cell expressing the same chimeric receptor. A transmembrane domain may be derived from a natural or recombinant source. When the source is natural, the domain may be derived from any membrane-bound protein or transmembrane protein. In one aspect, the transmembrane domain is capable of signaling to the intracellular domain as long as the chimeric receptor is bound to the target antigen. Transmembrane domains particularly used in the present invention may include at least the following transmembrane domains: e.g., alpha, beta or delta chains of a T-cell receptor, CD28, CD27, CD3ε, CD45, CD4, CD5, CD8, CD9, CD16, CD22, CD33, CD37, CD64, CD80, CD86, CD134, CD137, CD154 transmembrane domains. In some embodiments, the transmembrane domains may include at least the following transmembrane regions: e.g., KIRDS2, OX40, CD2, CD27, LFA-1 (CD1 la, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C transmembrane regions.

As used herein, “intracellular domain” (also referred to as intracellular region) includes intracellular signaling domains. The intracellular signaling domain is generally responsible for activation of at least one of the normal effector functions of the immune cell into which the chimeric receptor is introduced. The term “effector function” refers to a specialized function of a cell. The effector function of a T cell may be, for example, cytolytic activity or helper activity, including secretion of cytokines. Thus, the term “intracellular signaling domain” refers to the portion of a protein that transduces effector function signals and directs a cell to perform a specific function. Although the entire intracellular signaling domain may generally be used, in many cases it is not necessary to use the entire chain. As for using a truncated portion of an intracellular signaling domain, such a truncated portions may be used in place of the entire chain, so long as it transduces effector function signals. Thus, the term intracellular signaling domain means including a truncated portion of the intracellular signaling domain sufficient to transduce effector function signals.

It is well known that signals generated by TCR alone are not sufficient to fully activate a T cell, and secondary and/or costimulatory signals are also required. Thus, T cell activation may be said to be mediated by two distinct classes of cytoplasmic signaling sequences: those eliciting antigen-dependent primary activation via the TCR (primary intracellular signaling domain), and those acting in an antigen-independent manner to provide secondary or costimulatory signals (secondary cytoplasmic domain, e.g., costimulatory domain).

The term “stimulation” refers to the binding of a stimulatory molecule (e.g., a TCR/CD3 complex or CAR) to its homologous ligand (or, in the case of a CAR, a tumor antigen), thereby mediating a signaling event, such as but not limited to a primary response induced via signaling of the TCR/CD3 complex or via signaling of a suitable NK receptor or signaling domain of a CAR. Stimulation may mediate altered expression of certain molecules.

The term “stimulatory molecule” refers to a molecule expressed by an immune cell (e.g., T cell, NK cell, B cell) that provides a cytoplasmic signaling sequence modulating the activation of immune cells for at least some aspects of immune cell signaling pathways in a stimulatory manner. In one aspect, the signal is a primary signal initiated by, e.g., binding of a TCR/CD3 complex to a peptide-loaded MEW molecule, and which results in mediating T cell responses including, but not limited to, proliferation, activation, differentiation, and the like. A primary cytoplasmic signaling sequence (also referred to as “primary signaling domain”) that acts in a stimulatory manner may comprise signaling motifs what are known as immunoreceptor tyrosine-based activation motifs (ITAMs). Examples of ITAM-containing cytoplasmic signaling sequences particularly useful in the present invention include, but are not limited to, those derived from: CD3ζ, common FcRγ (FCER1G), FcγRIIa, FcRβ (FcEpsilon R1b), CD3γ, CD3δ, CD3ε, CD79a, CD79b, DAP10 and DAP12. In the specific CARs of the invention, the intracellular signaling domain in any one or more of the CARs of the invention comprises an intracellular signaling sequence, such as the primary signaling sequence of CD3-delta. In the specific CARs of the invention, the primary signaling sequence of CD3-delta is the equivalent residues from a human or non-human species such as mouse, rodent, monkey, ape, etc.

The term “costimulatory molecule” refers to a cognate binding partner on a T cell, and it specifically binds a costimulatory ligand, thereby mediating a costimulatory response of the T cell, including but not limited to proliferation. Costimulatory molecules are cell surface molecules other than antigen receptors or their ligands, and they promote an effective immune response. Costimulatory molecules include, but are not limited to, MEW class I molecules, BTLA and Toll ligand receptors, and OX40, CD27, CD28, CDS, ICAM-1, LFA-1 (CD11a/CD18), ICOS (CD278), and 4-1BB (CD137). Further examples of such costimulatory molecules include: CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11 a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1(CD226), SLAMF4(CD244, 2B4), CD84, CD96(Tactile), CEACAM1, CRTAM, Ly9(CD229), CD160(BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and ligands specifically binding to CD83.

A costimulatory intracellular signaling domain is the intracellular portion of a costimulatory molecule. Costimulatory molecules are represented by the following protein families: TNF receptor proteins, immunoglobulin-like proteins, cytokine receptors, integrins, signaling lymphocytic activation molecules (SLAM proteins), and NK cell receptors. Examples of such molecules include: CD27, CD28, 4-1BB (CD137), OX40, GITR, CD30, CD40, ICOS, BAFFR, HVEM, ICAM-1, lymphocyte function associated antigen-1 (LFA-1), CD2, CDS, CD7, CD287, LIGHT, NKG2C, NKG2D, SLAMF7, NKp80, NKp30, NKp44, NKp46, CD160, B7-H3, and ligands specifically binding to CD83, etc.

An intracellular signaling domain includes the entire intracellular portion or the entire natural intracellular signaling domain of a molecule, or a functional fragment or derivative thereof.

The term “4-1BB” refers to a member of the TNFR superfamily having an amino acid sequence as provided in GenBank Accession No. AAA62478.2, or equivalent residues from a non-human species such as mouse, rodent, monkey, ape, etc.; and “4-1BB costimulatory domain” is defined as amino acid residues 214-255 of GenBank Accession No. AAA62478.2, or equivalent residues from non-human species such as mouse, rodent, monkey, ape, etc. In one aspect, a “4-1BB costimulatory domain” is equivalent residues from a human or from a non-human species such as mouse, rodent, monkey, ape, etc.

The term “T cell receptor (TCR)” refers to a characteristic marker on the surface of all T cells, which binds to CD3 non-covalently to form a TCR-CD3 complex. The TCR is responsible for recognizing an antigen bound to a major histocompatibility complex molecule. TCR is a heterodimer composed of two different peptide chains, consisting of α and β peptide chains, each peptide chain may be divided into variable region (V region), constant region (C region), transmembrane region and cytoplasmic region; and is characterized by a short cytoplasmic region. The TCR molecule belongs to the immunoglobulin superfamily, and its antigen specificity exists in the V region; and the V region (Vα, Vβ) has three hypervariable regions (CDR1, CDR2, and CDR3); wherein CDR3 has the largest variation, which directly determines the antigen-binding specificity of TCR. When TCR recognizes the MHC-antigen peptide complex, CDR1 and CDR2 recognize and bind to the side wall of the antigen-binding groove of the MHC molecule, while CDR3 directly binds to the antigen peptide. TCRs are divided into two categories: TCR1 and TCR2; TCR1 is composed of two chains(γ and δ), and TCR2 is composed of two chains (α and (β).

The term “T cell fusion protein (TFP)” includes recombinant polypeptides derived from various polypeptides constituting the TCR, which are capable of binding to surface antigens on target cells and interacting with other polypeptides of the complete TCR complex, usually co-localized on the surface of T cells. TFP is composed of an antigen binding domain consisting of a TCR subunit and a human or humanized antibody domain, wherein the TCR subunit comprises at least part of the TCR extracellular domain, transmembrane domain, the stimulatory domain of the intracellular signaling domain of TCR intracellular domain; the TCR subunit is operably linked to the antibody domain, wherein the extracellular, transmembrane, and intracellular signaling domains of the TCR subunit are derived from CD3ε or CD3γ, and the TFP is integrated into TCRs expressed on T cells.

The term “T cell antigen coupler (TAC)” comprises three functional domains: 1. tumor targeting domain, comprising single chain antibody, designed ankyrin repeat protein (DARPin) or other targeting groups; 2. the extracellular domain, a single-chain antibody binding to CD3, thereby bringing the TAC receptor close to the TCR receptor; 3. the transmembrane domain and the intracellular domain of the CD4 co-receptor, wherein the intracellular domain is linked to the protein kinase LCK, which catalyzes the phosphorylation of immunoreceptor tyrosine activation motifs (ITAMs) of the TCR complex as an initial step of T cell activation.

The term “NF-kb (Nuclear factor kB)” is a member of the transcription factor family, and is the most important nuclear transcription factor in cells. It plays a central role in many transcriptional regulation of cell information mediated by cell stimuli, and is involved in the expression and regulation of a variety of genes, it is a hallmark of cell activation. NF-κB generally exists in the form of homo- or hetero-dimers. In resting cells, NF-KB dimers are dispersed in the cytoplasm through non-covalently binding to its inhibitory protein IKB, and many factors including endoplasmic reticulum stress may activate NF-κB, and after activation NF-kB enters the nucleus and binds to specific proteins on DNA modules, then induces the production of specific mRNAs, and finally transcribes, produces and releases various cytokines.

The term “AP-1 (activator protein 1)” is an intracellular transcriptional activator, which is a heterodimer composed of c-Fos and c-Jun. It responds to a variety of stimuli, including cytokines, growth factors, stress, bacterial and viral infections, by regulating gene expression; thus AP-1 controls many cellular processes, including differentiation, proliferation, and apoptosis. AP-1 upregulates the transcription of genes comprising the TPA DNA response element (TRE; 5′-TGAG/CTCA-3′). AP-1 heterodimer is formed by a leucine zipper, and binds to a gene through specific conserved sequence to initiate gene expression.

The term “nuclear factor of activated T cells” (NFATs) plays an important role in the transcriptional regulation of cytokine genes, and NFAT protein plays an important role in the transcriptional regulation of a variety of cytokines and cell surface receptors (for example, interleukin-2, interleukin-4, interleukin-5, interleukin-13, interferon-γ, tumor necrosis factor-α, GM-CSF, CD4OL and CTLA-4) that regulate important immune functions. The NFAT proteins discovered so far may be divided into five types: NFAT1, NFAT2, NFAT3, NFAT4 and NFAT5, wherein the activation of NFATc1-4 is dependent on the intracellular calcium signaling pathway.

The activation of NFAT proteins is regulated by a process including NFAT protein dephosphorylation, nuclear translocation, and DNA binding. In resting cells, phosphorylated NFAT proteins reside in the cytoplasm, and have lower DNA binding affinity. Various stimuli capable of triggering calcium mobilization may cause rapid dephosphorylation of NFAT proteins through a process regulated by the Ca2+/calmodulin-dependent protein phosphatase, i.e., calcineurin. Dephosphorylated NFAT proteins with an exposed nuclear localization signal are translocated into the nucleus, where they bind DNA with high affinity and regulate target gene transcription. In some embodiments, NFAT plays an important role in the transcriptional expression of cytokines during T cell activation. In some embodiments, IL7 is inducibly expressed by using an inducible promoter. In some embodiments, the inducible promoter is an NFAT promoter. In some embodiments, the coding sequence for IL7 is placed under the regulation of a minimal promoter containing an NFAT binding motif. In some particular embodiments, the IL2 minimal promoter containing 6 NFAT binding motifs is a promoter composed of 6 NFAT binding sites in tandem with a minimal promoter of IL2. In embodiments of the invention, when the receptor recognizes the target antigen, the activated TCR signal may activate NFAT in cells to bind to the NFAT binding motif in the promoter, thereby initiating transcription of IL7.

As used herein, the term “promoter” is defined as a DNA sequence recognized by a cell's synthetic machinery or introduced synthetic machinery necessary to initiate specific transcription of a polynucleotide sequence. A promoter is a DNA sequence which is recognized, bound and initiated the transcription by RNA polymerase. It comprises the conserved sequences required for RNA polymerase specific binding and transcription initiation.

A typical eukaryotic promoter consists of a minimal promoter and other cis-elements. The minimal promoter is essentially a TATA box region where RNA polymerase II (polll), TATA-binding protein (TBP) and TBP-associated factor (TAF) may bind to initiate transcription. Such sequence elements (e.g., enhancers) have been found to increase the overall expression level of adjacent genes, often in a position- and/or orientation-independent manner. The construction of chimeric promoters obtained by combining a minimal promoter with different cis-regulatory elements is described, for example, in U.S. Pat. No. 6,555,673.

In some embodiments, NFAT plays an important role in the transcription and expression of cytokines during T cell activation. With this in mind, the present inventors placed the coding sequences for cytokines under the regulation of a minimal promoter containing an NFAT binding motif. In addition, in order to further improve the specificity of cytokine-expressing CAR-T cells, the endogenous TCRα chain of CAR-GPC3T cells carrying NFAT-regulated cytokine-expressing genes may also be knocked out by gene editing technology, so as to eliminate the expression of cytokines induced by non-tumor targets antigens (such as non-GPC3 antigens) through the TCR/CD3 signaling pathway, so that only tumor target antigens specifically induce CAR-GPC3 T cells to express cytokines, such as IL7.

In an embodiment, the IL2 minimal promoter containing 6 NFAT binding motifs is a promoter composed of 6 NFAT binding sites (e.g., nucleic acid sequence represented by SEQ ID NO: 16) and the minimal promoter of IL2 in tandem (Hooijberg E, Bakker A Q, Ruizendaal J J, Spits H. NFAT-controlled expression of GFP permits visualization and isolation of antigen-stimulated primary human Tcells. Blood. 2000 Jul. 15; 96(2): 459-66), which may be used to regulate the expression of cytokines such as IL12 in T lymphocytes such as TCR-T (Zhang L, Kerkar S P, Yu Z, Zheng Z, Yang S, Restifo N P, Rosenberg S A, Morgan R A. Improving adoptive T cell therapy by targeting and controlling IL-12 expression to the tumor environment. Mol Ther. 2011 April; 19(4): 751-9).

The term “antibody” refers to a protein or polypeptide sequence derived from an immunoglobulin molecule that specifically binds an antigen. An antitibody may be polyclonal or monoclonal, multi-chain or single-chain, or an intact immunoglobulin, and may be derived from natural or recombinant sources. An antibody may be a tetramer of immunoglobulin molecules.

The term “antibody fragment” refers to at least a portion of an antibody that retains the ability to specifically interact (e.g., through binding, steric hindrance, stabilization/destabilization, steric distribution) with an epitope of an antigen. Examples of antibody fragments include, but are not limited to, Fab, Fab′, F(ab′)₂, Fv fragment, scFv, disulfide-linked Fvs (sdFv), Fd fragment consisting of VH and CH1 domains, linear antibody , single domain antibody (such as sdAb), multispecific antibody formed from antibody fragments (such as bivalent fragment comprising two Fab fragments linked by disulfide bonds at the hinge region), and isolated CDR or other epitope binding fragments of an antibody.

The term “scFv” refers to a fusion protein which comprises at least one antibody fragment comprising a variable region of a light chain and at least one antibody fragment comprising a variable region of a heavy chain, wherein the light and heavy chain variable regions are contiguous (for example, via a synthetic linker such as a short flexible polypeptide linker), and may be expressed as a single-chain polypeptide, and wherein the scFv retains the specificity of the intact antibody from which it is derived. Unless otherwise specified, as used herein, a scFv may have the VL and VH variable regions described in any order (e.g., with respect to the N-terminus and C-terminus of the polypeptide), the scFv may comprise a VL-linker-VH, or a VH-linker-VL.

The term “antibody heavy chain” refers to the larger of two polypeptide chains that are present in an antibody molecule in its naturally occurring configuration and generally determine the class to which the antibody belongs.

The term “antibody light chain” refers to the smaller of two polypeptide chains present in an antibody molecule in its naturally occurring configuration. Kappa (k) and lambda (l) light chains refer to the two major antibody light chain isotypes.

The term “recombinant antibody” refers to an antibody produced by using recombinant DNA technology, for example, an antibody expressed by phage or yeast expression system. The term should also be interpreted as referring to an antibody produced by synthesizing a DNA molecule encoding the antibody (and wherein the DNA molecule expresses the antibody protein) or the amino acid sequence of the specified antibody, wherein the DNA or amino acid sequence has been obtained by using recombinant DNA technology or well-known amino acid sequence technology available in the art.

The term “antigen” refers to a molecule that elicits an immune response. The immune response may involve antibody production or activation of a cell with specific immunocompetence or both. Those skilled in the art will understand that any macromolecule, including virtually all proteins or peptides, may serve as an antigen. In addition, an antigen may be derived from recombinant or genomic DNA. When the term is used herein, one of skill in the art will understand that it includes a protein or peptide encoded by any DNA of a nucleotide sequence or a partial nucleotide sequence encoding a protein that elicits an immune response. Furthermore, those skilled in the art will understand that an antigen need not to be encoded solely by the full-length nucleotide sequence of a gene. It will be apparent that the present invention includes, but is not limited to, using partial nucleotide sequences of more than one gene, and that these nucleotide sequences are arranged in various combinations to encode a polypeptide that elicits a desired immune response. Furthermore, those skilled in the art will understand that an antigen need not to be encoded by a “gene”. Obviously, an antigen may be produced synthetically, or may be derived from a biological sample, or may be a macromolecule other than polypeptide. Such biological samples may include, but are not limited to, a tissue sample, tumor sample, a cell or fluid having other biological components.

“Tumor antigen” refers to an antigen that is newly emerged or overexpressed during the development, progression of a hyperproliferative disease. In certain aspects, the hyperproliferative disorder of the invention refers to a cancer.

The tumor antigens of the present invention may be solid tumor antigens or hematological tumor antigens.

The tumor antigens of the present invention include, but are not limited to: thyroid-stimulating hormone receptor (TSHR); CD171; CS-1; C-type lectin-like molecule-1; ganglioside GD3; Tn antigen; CD19; CD20; CD22; CD 30; CD 70; CD 123; CD 138; CD33; CD44; CD44v7/8; CD38; CD44v6; B7H3 (CD276), B7H6; KIT (CD117); interleukin 13 receptor subunit alpha (IL-13Rα); interleukin 11 receptor alpha (IL-11Rα); prostate stem cell antigen (PSCA); prostate specific membrane antigen (PSMA); carcinoembryonic antigen (CEA); NY-ESO-1; HIV-1Gag; MART-1; gp100; tyrosinase; mesothelin; EpCAM; protease serine 21 (PRSS21); vascular endothelial growth factor receptor, vascular endothelial growth factor receptor 2 (VEGFR2); Lewis (Y) antigen; CD24; platelet-derived growth factor receptor f3 (PDGFR-β); stage-specific embryonic antigen-4 (SSEA-4); cell surface associated mucin 1 (MUC1), MUC6; epidermal growth factor receptor family and its mutants (EGFR, EGFR2, ERBB3, ERBB4, EGFRvIII); neural cell adhesion molecule (NCAM); carbonic anhydrase IX (CAIX); LMP2; ephrin A receptor 2 (EphA2); fucosyl GM1; sialyl Lewis adhesion molecule (sLe); ganglioside GM3; TGS5; high molecular weight melanoma-associated antigen (HMWMAA); o-acetyl GD2 ganglioside (OAcGD2); folate receptor; tumor endothelial marker 1 (TEM1/CD248); tumor endothelial marker 7-related (TEM7R); Claudin 6, Claudin18.2, Claudin18.1; ASGPR1; CDH16; 5T4; 8H9; αvβ6 integrin; B cell maturation antigen (BCMA); CA9; kappa light chain ; CSPG4; EGP2, EGP40; FAP; FAR; FBP; embryonic AchR; HLA-A1, HLA-A2; MAGEA1, MAGE3; KDR; MCSP; NKG2D ligand; PSC1; ROR1; Sp17; SURVIVIN; TAG72; TEM1; fibronectin; carcinoembryonic variant of tumor necrosis region; G protein-coupled receptor class C group 5-member D (GPRCSD); X chromosome open reading frame 61 (CXORF61); CD97; CD179a; anaplastic lymphoma kinase (ALK); polysialic acid; placenta-specific 1 (PLAC1); the hexose moiety of globoH glycoceramide (GloboH); mammary gland differentiation antigen (NY-BR-1); uroplakin 2 (UPK2); hepatitis A virus cell receptor 1 (HAVCR1); adrenergic receptor beta 3 (ADRB3); pannexin 3 (PANX3); G protein coupled receptor 20 (GPR20); lymphocyte antigen 6 complex locus K9 (LY6K); olfactory receptor 51E2 (OR51E2); TCRγ alternating reading frame protein (TARP); Wilms tumor protein (WT1); ETS translocation variant gene 6 (ETV6-AML); sperm protein 17 (SPA17); X antigen family member 1A (XAGE1); angiopoietin-binding cell surface receptor 2 (Tie2); melanoma cancer testis antigen-1 (MAD-CT-1); melanoma cancer testis antigen-2 (MAD-CT-2); Fos-associated antigen 1; p53 mutant; human telomerase reverse transcriptase (hTERT); sarcoma translocation breakpoint; apoptotic melanoma inhibitor (ML-IAP); ERG (transmembrane protease serine 2 (TMPRSS2) ETS fusion gene); N-acetylglucosaminyltransferase V (NA17); paired box protein Pax-3 (PAX3); androgen receptor; cyclin B 1; V-myc avian myelocytoma virus oncogene neuroblastoma-derived homolog (MYCN); Ras homolog family member C (RhoC); cytochrome P450 1B1 (CYP1B1); CCCTC-binding factor (zinc finger protein)-like (BORIS); squamous cell carcinoma antigen 3 recognized by T cells (SART3); paired box protein Pax-5 (PAX5); proacrosin-binding protein sp32 (OYTES1); lymphocyte-specific protein tyrosine kinase (LCK); A-kinase-anchored protein 4 (AKAP-4); synovial sarcoma X breakpoint 2 (SSX2); CD79a; CD79b; CD72; leukocyte-associated immunoglobulin-like receptor 1 (LAIR1); Fc fragment of IgA receptor (FCAR); leukocyte immunoglobulin-like receptor subfamily member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); glypican-3 (GPC3); Fc receptor-like 5 (FCRL5); immunoglobulin lambda-like polypeptide 1 (IGLL1).

The pathogen antigen is selected from the group consisting of: antigens of viruses, bacteria, fungi, protozoa, or parasites; the viral antigen is selected from the group consisting of:: cytomegalovirus antigen, Epstein-Barr virus antigen, human immunodeficiency virus antigen, or influenza virus antigen.

The term “tumor” refers to the broad category of disorders of hyperproliferative cell growth in vitro (e.g., transformed cells) or in vivo. Conditions that may be treated or prevented by the methods of the present invention include, for example, various neoplasms, including benign or malignant tumors, various hyperplasias, and the like. Tumors include but are not limited to: breast cancer, prostate cancer, leukemia, lymphoma, nasopharyngeal cancer, brain glioma, colon cancer, rectal cancer, renal cell carcinoma, liver cancer, non-small cell lung cancer, small intestine cancer, esophageal cancer, melanoma tumor, bone cancer, pancreatic cancer, skin cancer, head and neck cancer, uterine cancer, ovarian cancer, stomach cancer, testicular cancer, fallopian tube cancer, endometrial cancer, cervical cancer, vaginal cancer, thyroid cancer, parathyroid cancer, adrenal cancer, soft tissue sarcoma, urethral cancer, penile cancer, bladder cancer, ureteral cancer, renal pelvis cancer, central nervous system (CNS) tumor, hemangioma, spinal tumor, glioma, astrocytoma, pituitary adenoma, a combination of these cancers, and metastatic lesions of these cancers.

The term “transfected”, or “transformed”, or “transduced” refers to a process by which exogenous nucleic acid is transferred or introduced into a host cell. A “transfected”, or “transformed”, or “transduced” cell is a cell transfected, transformed or transduced with exogenous nucleic acid. The cells include primary subject cells and their progeny.

The term “specifically binding” means that an antibody or ligand binds to a binding partner (e.g., a tumor antigen) present in a sample, but substantially does not recognize or bind to other molecules in the sample.

“Refractory” as used herein refers to a disease (e.g., a tumor) that does not respond to a treatment. In some embodiments, a refractory tumor may be resistant to a treatment prior to or at initiation of the treatment. In other embodiments, a refractory tumor may be one that develops resistance to a treatment during the treatment period. In the present invention, refractory tumors include, but are not limited to, radiotherapy-insensitive, relapsed after radiotherapy, chemotherapy-insensitive, relapsed after chemotherapy, insensitive to CAR-T therapy, or relapsed after treatment. Refractory or relapsed malignancies may be treated with the treatment regimens described herein.

As used herein, “relapsed” means that signs and symptoms prior to the effective treatment reappear in a patient after a period of improvement, e.g., the preceding effective tumor treatment.

The terms “individual” and “subject” have equivalent meanings herein, and may be a human or an animal from other species.

The term “enhance” refers to allowing a subject or tumor cell to improve its ability to respond to the treatments disclosed herein. For example, an enhanced response may comprise 5%, 10%, 15%, 20%, 25%, 30%, 35%, 40%, 45%, 50%, 55%, 60%, 65%, 70%, 75%, 80%, 85%, 90%, 95% or 98% or more increase in responsiveness. As used herein, “enhance” may also refer to increasing the number of subjects that respond to a treatment, e.g., immune effector cell therapy. For example, an enhanced response may refer to the total percentage of subjects responding to a treatment, wherein the percentage is 5%, 10%, 15%, 20%, 25%, 30%, 35%, 40%, 45%, 50%, 55% %, 60%, 65%, 70%, 75%, 80%, 85%, 90%, 95%, or 98%, or more.

In one aspect, the treatment is determined by a clinical outcome; e.g., by an increase, enhancement or prolongation of the anti-tumor activity of T cells; an increase in the number of anti-tumor T cells or activated T cells compared to the number before treatment, promotion of IFN-γ secretion, or a combination thereof. In another aspect, the clinical outcome is tumor regression; tumor shrinkage; tumor necrosis; anti-tumor response by the immune system; tumor expansion, recurrence or spread, or a combination thereof. In an additional aspect, the therapeutic effect is predicted by the presence of T cells, the presence of genetic markers indicative of T cell inflammation, promotion of IFN-gamma secretion, or a combination thereof.

Immune effector cells as disclosed herein may be administered to an individual by various routes including, for example, orally or parenterally, e.g., intravenous, intramuscular, subcutaneous, intraorbital, intracapsular, intraperitoneal, intrarectal, intracisternal, intratumoral, intranasally, intradermally, or passive or facilitated absorption through the skin by using, for example, a skin patch or transdermal iontophoresis, respectively.

The total amount of agent to be administered in practicing the methods of the present invention may be administered to a subject by bolus injection as a single dose or by infusion over a relatively short period of time, or may be administered by using a graded treatment regimen, wherein multiple doses may be administed over an extended period of time. Those of skill in the art will know that the amount of the composition for treating a pathological condition in a subject depends on many factors, including the age and general health of the subject, as well as the route of administration and the number of treatments to be administered. Taking these factors into account, the technician will adjust the specific dose as needed. In general, Phase I and Phase II clinical trials are initially used to determine the formulation of the composition, as well as the route and frequency of administration.

Ranges: throughout this disclosure, various aspects of the invention may be presented in a range format. It should be understood that the description in range format is merely for convenience and brevity, and should not be regarded as an inexorable limitation on the scope of the invention. Accordingly, the description of a range should be considered to specifically disclose all possible subranges, as well as individual numerical values within that range. For example, the description of a range, such as from 1 to 6, should be considered to specifically disclose subranges such as 1 to 3, 1 to 4, 1 to 5, 2 to 4, 2 to 6, 3 to 6, etc., as well as individual values within that range, such as 1, 2, 2.7, 3, 4, 5, 5.3 and 6. As another example, a range such as 95-99% identity includes ranges having 95%, 96%, 97%, 98%, or 99% identity, and includes subranges such as 96-99%, 96-98%, 96-97%, 97-99%, 97-98%, and 98-99% identity. This applies regardless of the width of the range.

Based on the present disclosure, those of skill in the art should appreciate that many modifications or changes may be made in the particular embodiments disclosed, and still obtain a like or similar result without departing from the spirit and scope of the invention. The scope of the present invention is not limited to the particular embodiments described herein, which are intended only as illustrations of various aspects of the invention, and functionally equivalent methods and components are within the scope of this invention.

When the CAR-T cells inducibly expressing IL7, or IL7 and CCL21, or IL7 and CCL19 are used in a subject, the corresponding species may be selected. For example, when they are used in mice, murine-derived IL7 and CCL21, or murine-derived IL7 and CCL19 are selected, and the elements for constructing CAR such as transmembrane domain, intracellular domain, etc. may also be selected from murine origin. When the subject is human, elements of human-derived IL7 and CCL21, or human-derived IL7 and CCL19, and human-derived CAR are preferred. In some embodiments, the sequence of the CAR used may be represented by SEQ ID NO: 13, 14, 15, 23, 24, 25, or 26.

In some embodiments, the cells of the present invention may be used in combination with a chemotherapeutic agent when they are used in tumor therapy.

The term “GPC3” is Glypican-3 (Gene Nos. NP 004475.1, NM 004484.4) (Glypican-3, also known as DGSX, GTR2-2, MXR7, OCI-5, SDYS, SGB, SGBS or SGBS1), and it is a cell surface protein belonging to the heparan sulfate proteoglycan family. The GPC3 gene encodes a precursor core protein of about 70-kDa that may be cleaved by furin to produce a soluble amino-terminal (N-terminal) peptide of about 40-kDa that may enter the blood, and a membrane-bound carboxyl-terminal (C-terminal) peptide of about 30-kDa that comprises two heparan sulfate (HS) sugar chains. The GPC3 protein is attached to the cell membrane through a glycosylphosphatidylinositol (GPI) anchor. The sequence of human GPC3 protein is represented by SEQ ID NO.35.

The term “GPC3” includes any post-translationally modified variant, isoform and interspecies homolog of GPC3 that is naturally expressed by a cell or expressed by a cell transfected with the GPC3 gene.

The term “GPC3 variant” shall include: (i) GPC3 splice variants, (ii) GPC3 post-translationally modified variants, in particular variants differing in the N-glycosylation state, (iii) GPC3 conformational variants, (iv) GPC3 and homo/heterotype-associated variants located on the cell surface, (v) GPC3 cancer-associated variants and GPC3 non-cancer-associated variants.

The chimeric antigen receptor polypeptides of the present invention may be selected from the following sequential linkages:

extracellular antigen binding region-CD8 transmembrane region-4-1BB-CD3ζ,

extracellular antigen binding region-CD8 transmembrane region-CD28b-CD3ζ,

extracellular antigen binding region-CD28a-CD28b-CD3ζ,

extracellular antigen binding region-CD28a-CD28b-4-1BB-CD3ζ,

and a combination thereof, wherein CD28a in the related chimeric antigen receptor protein represents the transmembrane region of the CD28 molecule, and CD28b represents the intracellular signal region of the CD28 molecule. The present invention also encompasses nucleic acids encoding such chimeric antigen receptors. The present invention also relates to variants of the above-mentioned polynucleotides, which encode a polypeptide or a fragment, analog and derivative thereof having the same amino acid sequence as that of the present invention.

The present invention also provides a vector comprising the nucleic acid of the above-mentioned chimeric antigen receptor. The present invention also encompasses a virus comprising the above-described vector. The virus of the present invention includes the packaged infectious virus, and also includes the virus to be packaged which comprises the necessary components for packaging it as an infectious virus. Other viruses and their corresponding plasmid vectors known in the art that may be used for transducing an exogenous gene into an immune effector cells may also be used in the present invention.

The present invention also provides a chimeric antigen-modified immune effector cell, which is transduced with a nucleic acid encoding the chimeric antigen receptor, or transduced with the above-mentioned recombinant plasmid comprising the nucleic acid, or a virus comprising the plasmid. Conventional nucleic acid transduction methods in the art, including non-viral and viral transduction methods, may be used in the present invention. Non-viral-based transduction methods include electroporation and transposon methods. Recently, the Nucleofector nucleofection instrument developed by Amaxa may directly introduce an exogenous gene into the nucleus to obtain efficient transduction of a target gene. In addition, the transduction efficiency based on the Sleeping Beauty transposon (Sleeping Beauty system) or the PiggyBac transposon and other transposon systems is greatly improved as compared with ordinary electroporation. The combined application of nucleofector transfection instrument and Sleeping Beauty transposon system has been reported [Davies J K., et al. Combining CD19 redirection and alloanergization to generate tumor-specific human T cells for allogeneic cell therapy of B-cell malignancies. Cancer Res, 2010, 70(10): OF1-10.], this method has both high transduction efficiency and site-specific integration of a target gene. In one embodiment of the present invention, a transduction method for realizing chimeric antigen receptor gene-modified immune effector cells is a virus-based transduction method such as a retroviral or lentiviral transduction method. The method has the advantages including high transduction efficiency, stable expression of exogenous genes, and shortening the time period for in vitro cultured immune effector cells to reach a mount of clinical level. On the surface of the transgenic immune effector cell, the transduced nucleic acid is expressed on its surface through transcription and translation. By in vitro cytotoxicity experiments on various cultured tumor cells, it is proved that the chimeric antigen-modified immune effector cells of the present invention have a highly specific tumor cell killing effect (also known as cytotoxicity), and may survive effectively in tumor tissues. Therefore, the nucleic acid encoding the chimeric antigen receptor of the present invention, the plasmid comprising the nucleic acid, the virus comprising the plasmid, and the transgenic immune effector cells transduced with the above nucleic acid, plasmid or virus may be effectively used for tumor immunotherapy.

The chimeric antigen-modified immune effector cells of the present invention may also express another chimeric receptor other than the above-mentioned chimeric receptor, the receptor does not contain CD3ζ, but contains the intracellular signaling domain of CD28, the intracellular signaling domain of CD137, or a combination of the two.

The chimeric antigen receptor-modified immune effector cells of the present invention may be applied to the preparation of a pharmaceutical composition or a diagnostic reagent. In addition to comprising an effective amount of the immune cells, the composition may also comprise a pharmaceutically acceptable carrier. The term “pharmaceutically acceptable” means that the molecular entities and compositions do not produce infaust, allergic or other adverse reactions when properly administered to animals or humans.

Specific examples of some substances which may be used as pharmaceutically acceptable carriers or components thereof are: carbohydrates such as lactose, glucose and sucrose; starches such as corn starch and potato starch; cellulose and its derivatives such as carboxymethyl cellulose sodium, ethyl cellulose and methyl cellulose; tragacanth powder; malt; gelatin; talc; solid lubricants such as stearic acid and magnesium stearate; calcium sulfate; vegetable oils such as peanut oil, cottonseed oil, sesame oil, olive oil, corn oil, and cocoa butter; polyols, such as propylene glycol, glycerol, sorbitol, mannitol, and polyethylene glycol; alginic acid; emulsifiers such as Tween®; wetting agents such as sodium lauryl sulfate; coloring agents; flavoring agents; tableting agents; stabilizers; antioxidants; preservatives; pyrogen-free water; isotonic saline solutions and phosphate buffers, etc.

The composition of the present invention may be prepared into various dosage forms according to needs, and may be administered to a patient at a beneficial dose determined by a physician according to factors such as the type, age, weight and general disease state of the patient, and the mode of administration. The mode of administration may be injection or other therapeutic methods.

The present invention will be further described below in conjunction with particular Examples. It should be understood that these Examples are only used to illustrate the present invention, and not to limit the scope of the present invention. As for an experimental method not stating specific conditions in the following examples, generally it may be performed according to conventional conditions such as those described in J. Sambrook et al., Molecular Cloning Laboratory Guide, 3rd Edition, Science Press, 2002, or as suggested by the manufacturer.

As for exemplary antigen receptors of the present invention including CARs, and methods for engineering and introducing a receptor into a cell, referring to, e.g., those disclosed in Chinese patent application publication Nos.: CN107058354A, CN107460201A, CN105194661A, CN105315375A, CN105713881A, CN106146666A, CN106519037A, CN106554414A, CN105331585A, CN106397593A, CN106467573A, CN104140974A, CN108884459A, CN107893052A, CN108866003A, CN108853144A, CN109385403A, CN109385400A, CN109468279A, CN109503715A, CN109908176A, CN109880803A, CN110055275A, CN110123837A, CN110438082A, and CN110468105A; and International patent application publication Nos.: WO2017186121A1, WO2018006882A1, WO2015172339A8, WO2018/018958A1, WO2014180306A1, WO2015197016A1, WO2016008405A1, WO2016086813A1, WO2016150400A1, WO2017032293A1, WO2017080377A1, WO2017186121A1, WO2018045811A1, WO2018108106A1, WO2018/219299, WO2018/210279, WO2019/024933, WO2019/114751, WO2019/114762, WO2019/141270, WO2019/149279, WO2019/170147A1, WO2019/210863, and WO2019/219029.

### EXAMPLE 1

### Construction of T Cells Expressing Chimeric Antigen Receptors

Exemplarily, in this example, GPC3 is selected as the target of CAR-T cells, and the preparation method is performed according to conventional CAR-T cell preparation methods in the art.

Using conventional molecular biology methods in the art, the scFv used in this example is an antibody targeting human GPC3 with a nucleic acid sequence represented by SEQ ID NO: 27, and the chimeric antigen receptor used in this example is a second-generation chimeric antigen receptor with the transmembrane domain of CD8 (the nucleic acid sequence is represented by SEQ ID NO: 17), the intracellular signaling domain of CD137 (the nucleic acid sequence is represented by SEQ ID NO: 36), and the intracellular segment CD3ζ signaling domain of CD3 (the nucleic acid sequence is represented by SEQ ID NO: 19).

Using pRRLSIN-cPPT.EF-1α as a vector, a lentiviral plasmid pRRLSIN-GPC3-BBZ expressing the second-generation chimeric antigen receptor is constructed. The nucleic acid sequence of GPC3-BBZ comprises CD8α signal peptide (SEQ ID NO: 20), scFv (SEQ ID NO: 27), CD8α hinge region (SEQ ID NO: 21), CD8 transmembrane domain (SEQ ID NO: 17), CD137 intracellular signaling domain (SEQ ID NO: 36), and CD3 intracellular segment CD3ζ signaling domain (SEQ ID NO: 19).

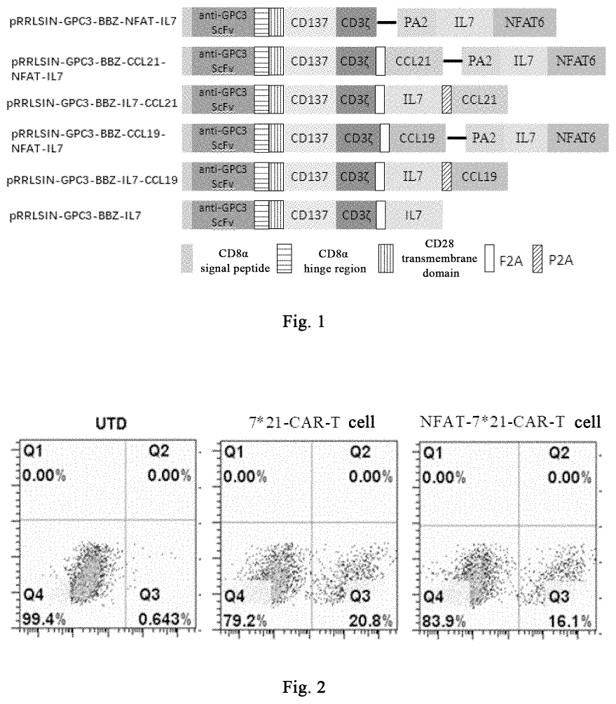

The gene of NFAT6-IL7 is inserted on the basis of the pRRLSIN-GPC3-BBZ plasmid to construct a lentiviral plasmid pRRLSIN-GPC3-BBZ-NFAT-IL7 that expresses CAR and regulates the expression of IL7 (the plasmid map is shown in FIG. 1). The nucleic acid sequence of NFAT6-IL7 comprises NFAT6 (SEQ ID NO: 16) and IL7 (SEQ ID NO: 28).

The gene of F2A-CCL21-NFAT6-IL7 is inserted on the basis of the pRRLSIN-GPC3-BBZ plasmid to construct the lentiviral plasmid pRRLSIN-GPC3-BBZ-CCL21-NFAT-IL7 that expresses CAR, constitutively expresses CCL21, and regulates the expression of IL7 (the plasmid map is shown in FIG. 1). The nucleic acid sequence of F2A-CCL21-NFAT6-IL7 comprises F2A (SEQ ID NO: 6), CCL21 (SEQ ID NO: 29), NFAT6 (SEQ ID NO: 16), and IL7 (SEQ ID NO: 28).

The gene of F2A-IL7-P2A-CCL21 is inserted on the basis of the pRRLSIN-GPC3-BBZ plasmid to construct the lentiviral plasmid pRRLSIN-GPC3-BBZ-CCL21-IL7 that expresses CAR, IL7, and CCL21 (the plasmid map is shown in FIG. 1). The nucleic acid sequence of F2A-IL7-P2A-CCL21 comprises F2A (SEQ ID NO: 6), IL7 (SEQ ID NO: 28), P2A (SEQ ID NO: 5), and CCL21 (SEQ ID NO: 29).

The gene of F2A-CCL19-NFAT6-IL7 is inserted on the basis of the pRRLSIN-GPC3-BBZ plasmid to construct the lentiviral plasmid pRRLSIN-GPC3-BBZ-CCL19-NFAT-IL7 that expresses CAR, constitutively expresses CCL19, and regulates the expression of IL7 (the plasmid map is shown in FIG. 1). The nucleic acid sequence of F2A-CCL19-NFAT6-IL7 comprises F2A (SEQ ID NO: 6), CCL19 (SEQ ID NO: 30), NFAT6 (SEQ ID NO: 16), and IL7 (SEQ ID NO: 28).

The gene of F2A-IL7-P2A-CCL19 is inserted on the basis of pRRLSIN-GPC3-BBZ plasmid to construct the lentiviral plasmid pRRLSIN-GPC3-BBZ-CCL19-IL7 that expresses CAR, IL7, and CCL19 (the plasmid map is shown in FIG. 1). The nucleic acid sequence of F2A-IL7-P2A-CCL19 comprises F2A (SEQ ID NO: 6), IL7 (SEQ ID NO: 28), P2A (SEQ ID NO: 5), and CCL19 (SEQ ID NO: 30).

The gene of F2A-IL7 is inserted on the basis of pRRLSIN-GPC3-BBZ plasmid to construct a lentiviral plasmid pRRLSIN-GPC3-BBZ-IL7 that expresses CAR and IL7 (the plasmid map is shown in FIG. 1). The nucleic acid sequence of F2A-IL7 comprises F2A (SEQ ID NO: 6) and IL7 (SEQ ID NO: 28).

PRRLSIN-GPC3-BBZ-NFAT-IL7, PRRLSIN-GPC3-BBZ-IL7, PRRLSIN-GPC3-BBZ-CCL21-NFAT-IL7, PRRLSIN-GPC3-BBZ-IL7-CCL21, PRRLSIN-GPC3-BBZ-CCL19-NFAT-IL7, and PRRLSIN-GPC3-BBZ-IL7-CCL19 are respectively transfected into 293T cells to obtain lentiviruses NFAT-IL7-BBZ, IL7-BBZ, NFAT-IL7-CCL21-BBZ, IL7-CCL21-BBZ, NFAT-IL7-CCL19-BBZ, and IL7-CCL19-BBZ.

Human peripheral blood PBMC cells are isolated, and after culture and activation, the resulting lentiviruses NFAT-IL7-BBZ, IL7-BBZ, NFAT-IL7-CCL21-BBZ, IL7-CCL21-BBZ, NFAT-IL7-CCL19-BBZ, and IL7-CCL19-BBZ are respectively used to infect T cells to obtain NFAT-IL7-CAR-T cells, IL7-CAR-T cells, NFAT-7*21-CAR-T cells, 7*21-CAR-T cells, NFAT-7*19-CAR-T cells, and 7*19-CAR-T cells.

The results of the positive rate of NFAT-7*21-CAR-T cells and 7*21-CAR-T cells are shown in FIG. 2, the positive rate of UTD is 0.643%; the positive rate of 7*21-CAR-T cells is 20.8%, and the positive rate of NFAT-7*21-CAR-T cells is 16.1%.

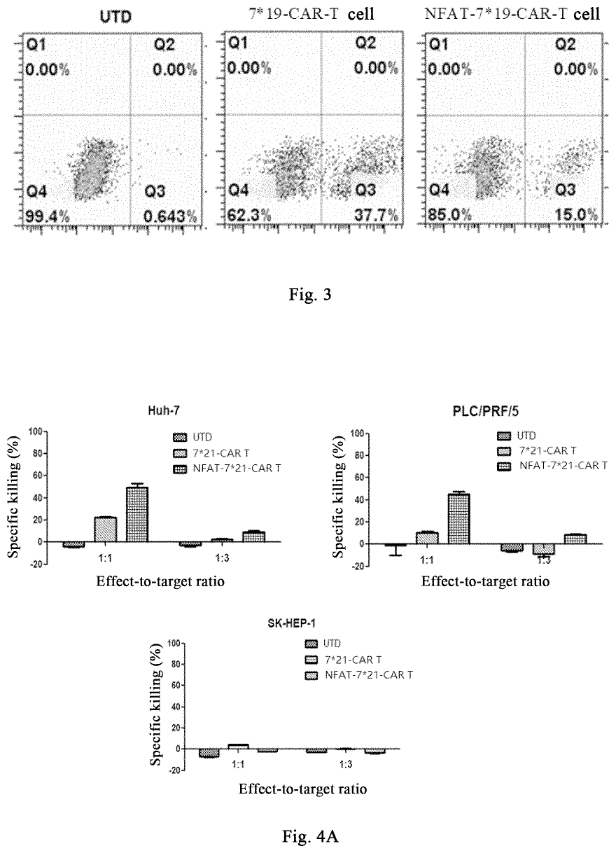

The results of the positive rate of NFAT-7*19-CAR-T cells and 7*19-CAR-T cells are shown in FIG. 3, the positive rate of UTD is 0.643%; the positive rate of 7*19-CAR-T cells is 37.7%, and the positive rate of NFAT-7*19-CAR-T cells is 15.0%.

### EXAMPLE 2

### In Vitro Cytotoxicity Assay

CytoTox 96 non-radioactive cytotoxicity detection kit (Promega) is used, for specific methods, referring to the instructions of CytoTox 96 non-radioactive cytotoxicity detection kit.

Effector Cells: UTD cells, NFAT-7*21-CAR-T cells, 7*21-CAR-T cells, NFAT-7*19-CAR-T cells and 7*19-CAR-T cells are respectively inoculated in a 96-well plate at an effect-to-target ratio (E:T) of 3:1, 1:1, or 1:3.

Target Cells: 50 μL of 1×10⁵/mL GPC3-positive human hepatoma Huh-7 cells and PLC/PRF/5 cells, and GPC3-negative human hepatoma SK-HEP-1 cells are respectively inoculated into a corresponding 96-well plate.

Five duplicate wells are set in each group, and the culture plates are placed in a cell incubator for 18 h.

The settings of each experimental group and each control group are as follows: Experimental group: each target cell+different CAR-T cells; Control group 1: maximum LDH release of target cells; Control group 2: spontaneous LDH release of target cells; Control group 3: spontaneous LDH release of effector cells. The calculation formula is: % cytotoxicity=[(Experimental group−effector cell spontaneous group−target cell spontaneous group)/(target cell maximum group−target cell spontaneous group)]*100.

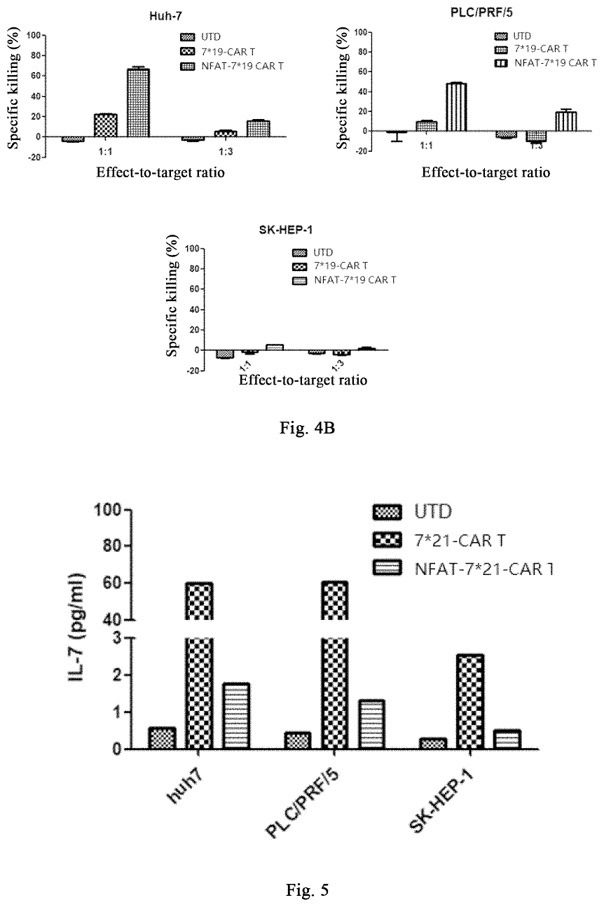

The experimental results are shown in FIGS. 4A and 4B. As for the GPC3-positive Huh7 and PLC/PRF/5 cells, when the effect-to-target ratio is 1:1 or 1:3, NFAT-7*21-CAR-T cells regulated by NFAT and NFAT-7*19-CAR-T cells regulated by NFAT show more excellent cell killing.

### EXAMPLE 3

### In Vitro Cytokine Assay

Human hepatoma cells Huh-7, PLC/PRF/5, and SK-HEP-1 are respectively used as target cells, and incubated in a system of 1*10^4 cells/well, E:T=3:1, for a total of 200 μl for 24 h. Then the supernatants are taken for detection.

The detection results of NFAT-7*21-CAR-T cells and 7*21-CAR-T cells are shown in FIG. 5. Co-incubated with GPC3-positive Huh-7 and PLC/PRF/5 cells, the secreted IL-7 may be detected in the 7*21-CAR-T group, and the secretion amount is higher; while the secretion amount of IL-7 regulated by NFAT in the NFAT-7*21-CAR-T group is lower, but higher than that in the UTD control group.

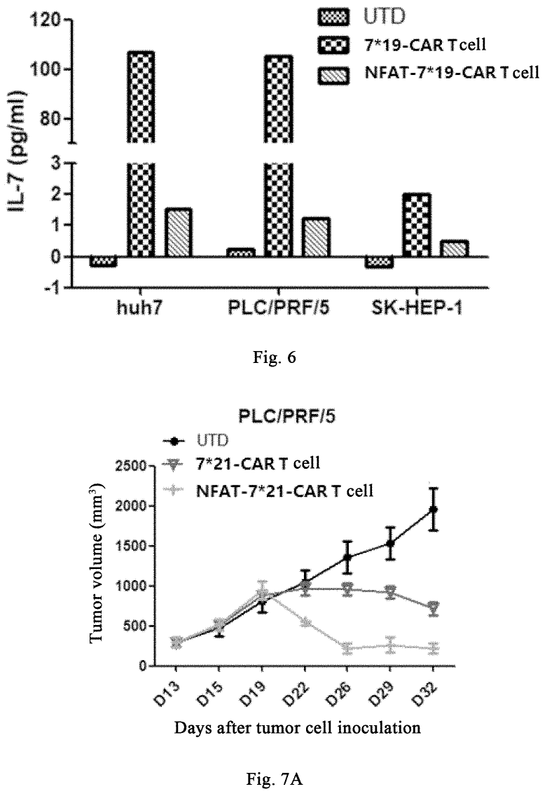

The detection results of NFAT-7*19-CAR-T cells and 7*19-CAR-T cells are shown in FIG. 6. Co-incubated with GPC3-positive Huh-7 and PLC/PRF/5 cells, the secreted IL-7 may be detected in the 7*19-CAR-T group, and the secretion amount is higher; while the secretion amount of IL-7 regulated by NFAT in the NFAT-7*19-CAR-T group is lower, but higher than that in the UTD control group.

### EXAMPLE 4

### In Vivo Killing of the NPG Mouse Subcutaneous Xenograft Model

Tumor Block Inoculation: 3×10⁶ liver cancer cells PLC/PRF/5 are respectively inoculated subcutaneously in the right axilla of female NPG mice, and they are divided into 5 groups with 6 mice in each group. The vaccination day is recorded as D0.

Injection of CAR-T: Day 13 after subcutaneous inoculation of tumor tissue, the average tumor volume is about 300 mm³. The CAR-T cells prepared in Example 1 are injected at the injection dose of 2.0×10⁶/mouse.

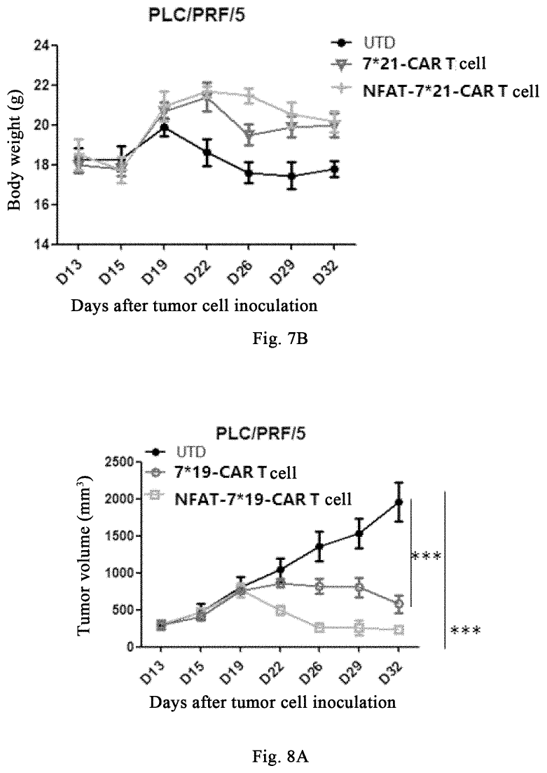

The tumor inhibition results of NFAT-7*21-CAR-T cells and 7*21-CAR-T cells are shown in FIG. 7A. For example, 19 days after CAR-T injection (Day 32), compared with the UTD group, the tumor inhibition rate of each group respectively is: 7*21-CAR-T cells: 63.44%; NFAT-7*21-CAR-T cells: 88.60%. At the same time, the changes in body weight of the mice in each group are detected, and the results are shown in FIG. 7B, and there is no significant change in body weight.

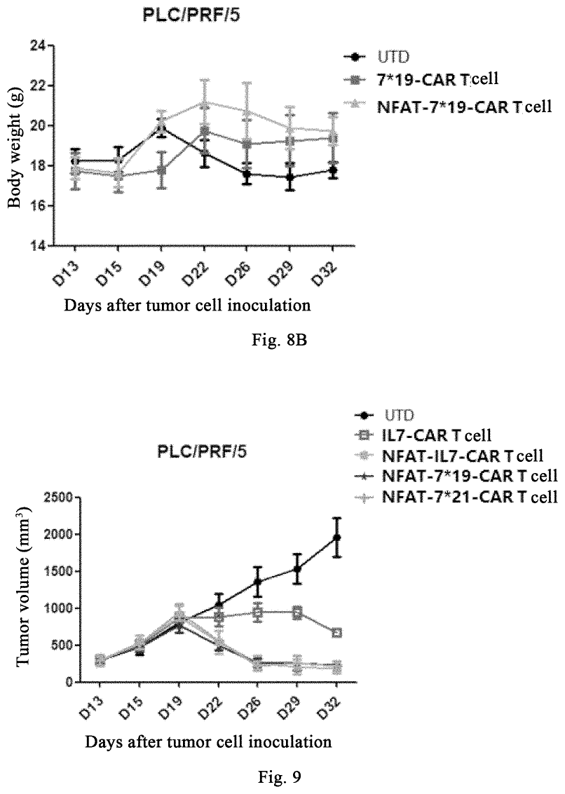

The tumor inhibition results of NFAT-7*19-CAR-T cells and 7*19-CAR-T cells are shown in FIG. 8A. 19 days after CAR T injection (Day 32), compared with the UTD group, the tumor inhibition rate of each group respectively is: 7*19-CAR-T cells: 70.45%; NFAT-7*19-CAR-T cells: 88.17%. At the same time, the changes in the body weight of the mice in each group are detected, and the results are shown in FIG. 8B, and there is no significant change in the body weight of the mice.

The tumor inhibition results of NFAT-IL7-CAR-T cells and IL7-CAR-T cells are shown in FIG. 9. Compared with IL7-CAR that constitutively expresses IL7, the tumor inhibition rate of NFAT-IL7-CAR-T cells induced by NFAT to regulate IL7 is significantly improved, and the tumor inhibition rate of NFAT-7*21-CAR-T or NFAT-7*19-CAR-T cells induced by NFAT to regulate IL7 and co-express chemokines (such as CCL21 or CCL19) is significantly improved.

Exemplary, CAR-T cells targeting GPC3 are selected in the above Examples, it should be understood that selection of CAR-T cells acting on other targets also has the same effect, such as claudin18.2, EGFR, EGFRvIII, CD19, BCMA, etc. The antibody used may be a mouse antibody or a humanized antibody, and the transmembrane domain and the intracellular domain may also be of different species, such as human, according to different purposes.

Exemplary, although CAR-T cells are used in the above Examples, the T cells may also express other cytokines enhancing the function of CAR-T cells, such as CAR-T cells co-expressing CAR and type I interferon, CAR-T cells co-expressing CAR and PD-1, etc. As an example, although CAR-T cells are used in the above Examples, other immune cells may also be selected, such as NK cells, NK-T cells, and specific subtypes of the immune cells may also be selected, such as γ/δT cells, etc.

The sequences used in the present invention are summarized in the following table:

| SEQ | | |
|---|---|---|
| ID | | |
| NO. | Names | Sequences |
| 1 | Human IL7 | Mfhvsfryifglpplilvllpvassdcdiegkdgkqyesvlmvsidqlldsmkeigsnclnnefnffkrhicdanke |
| | amino acid | gmflfraarklrqflkmnstgdfdlhllkvsegttillnctgqvkgrkpaalgeaqptksleenkslkeqkklndlc |
| | sequence | flkrllqeiktcwnkilmgtkeh |
| 2 | Murine IL7 | Atgttccatgtttcttttagatatatctttggaattcctccactgatccttgttctgctgcctgtcacatcatctga |
| | nucleotide | gtgccacattaaagacaaagAaggtaaagcatatgagagtgtactgatgatcagcatcgatgaattggacaaaatga |
| | sequence | caggaactgatagtaattgcccgaataatgaaccAaacttttttagaaaacatgtatgtgatgatacaaaggaagct |
| | | gcttttctaaatcgtgctgctcgcaagttgaagcaatttcttaaaatgaatatCagtgaagaattcaatgtccactt |
| | | actaacagtatcacaaggcacacaaacactggtgaactgcacaagtaaggaagaaaaaaacgtaaaggaacagaaaa |
| | | agaatgatgcatgtttcctaaagagactactgagagaaataaaaacttgttggaataaaattttgaagggcagtata |
| 3 | Minimal IL2 | Acattttgacacccccataatatttttccagaattaacagtataaattgcatctcttgttcaagagttccctatcac |
| | promoter | tctctttaatcactactcacagtaacctcaactcctg |
| 4 | Nucleic acid | GGAGGAAAAACTGTTTCATACAGAAGGCGTGGAGGAAAAACTGTTTCATACAG |
| | sequence of | AAGGCGTGGAGGAAAAACTGTTTCATACAGAAGGCGTCAATTGTCCTCGACGG |
| | NFAT-IL7 | AGGAAAAACTGTTTCATACAGAAGGCGTGGAGGAAAAACTGTTTCATACAGAA |
| | | GGCGTGGAGGAAAAACTGTTTCATACAGAAGGCGTCAATTGTCCCGGGACATT |
| | | TTGACACCCCCATAATATTTTTCCAGAATTAACAGTATAAATTGCATCTCTTGT |
| | | TCAAGAGTTCCCTATCACTCTCTTTAATCACTACTCACAGTAACCTCAACTCCT |
| | | GCATATGACCTAGCGCTACCGGTCGCCACCATGTTCCATGTTTCTTTTAGGTAT |
| | | ATCTTTGGACTTCCTCCCCTGATCCTTGTTCTGTTGCCAGTAGCATCATCTGAT |
| | | TGTGATATTGAAGGTAAAGATGGCAAACAATATGAGAGTGTTCTAATGGTCA |
| | | GCATCGATCAATTATTGGACAGCATGAAAGAAATTGGTAGCAATTGCCTGAA |
| | | TAATGAATTTAACTTTTTTAAAAGACATATCTGTGATGCTAATAAGGAAGGTA |
| | | TGTTTTTATTCCGTGCTGCTCGCAAGTTGAGGCAATTTCTTAAAATGAATAGC |
| | | ACTGGTGATTTTGATCTCCACTTATTAAAAGTTTCAGAAGGCACAACAATAC |
| | | TGTTGAACTGCACTGGCCAGGTTAAAGGAAGAAAACCAGCTGCCCTGGGTG |
| | | AAGCCCAACCAACAAAGAGTTTGGAAGAAAATAAATCTTTAAAGGAACAGA |
| | | AAAAACTGAATGACTTGTGTTTCCTAAAGAGACTATTACAAGAGATAAAAAC |
| | | TTGTTGGAATAAAATTTTGATGGGCACTAAAGAACAC |
| 5 | Nucleic acid | gctactaacttcagcctgctgaagcaggctggagacgtggaggagaaccctggacct |
| | sequence of | |
| | P2A | |
| 6 | Nucleic acid | Gtgaaacagactttgaattttgaccttctgaagttggcaggagacgttgagtccaaccctgggccc |
| | sequence of | |
| | F2A | |
| 7 | Amino acid | Maqslalsllilvlafgiprtqgsdggaqdcclkysqrkipakvvrsyrkqepslgcsipailflprkrsqaelcad |
| | sequence of | pkelwvqqlmqhldktpspqkpaqgcrkdrgasktgkkgkgskgckrtersqtpkgp |
| | human CCL21 | |
| 8 | Nucleotide | Atggctcagatgatgactctgagcctccttagcctggtcctggctctctgcatcccctggacccaaggcagtgatgg |
| | sequence of | agggggtcaggaCtgctgccttaagtacagccagaagaaaattccctacagtattgtccgaggctataggaagcaag |
| | murine | aaccaagtttaggctgtcccatcccGgcaatcctgttctcaccccggaagcactctaagcctgagctatgtgcaaac |
| | CCL21a | cctgaggaaggctgggtgcagaacctgatgcgccgcCtggaccagcctccagccccagggaaacaaagccccggctg |
| | | caggaagaaccggggaacctctaagtctggaaagaaaggaaagggctccaagggctgcaagagaactgaacagacac |
| | | agccctcaagagga |
| 9 | Nucleotide | Atggctcagatgatgactctgagcctccttagcctggtcctggctctctgcatcccctggacccaaggcagtgatgg |
| | sequence of | agggggacaggActgctgccttaagtacagccagaagaaaattccctacagtattgtccgaggctataggaagcaag |
| | murine | aaccaagtttaggctgtcccatccCggcaatcctgttcttaccccggaagcactctaagcctgagctatgtgcaaac |
| | CCL21b | cctgaggaaggctgggtgcagaacctgatgcgccgCctggaccagcctccagccccagggaaacaaagccccggctg |
| | | caggaagaaccggggaacctctaagtctggaaagaaaggaaagggctccaagggctgcaagagaactgaacagacac |
| | | agccctcaagagga |
| 10 | Amino acid | EVQLVQSGAEVKKPGASVKVSCKASGYTFSDYEMHWVRQAPGQGLEWMGAIHPGS |
| | sequence of | GDTAYNQRFKGRVTITADKSTSTAYMELSSLRSEDTAVYYCARFYSYAYWGQGTLV |
| | GPC3-scFV | TVSAGGGGSGGGGSGGGGSDIVMTQTPLSLPVTPGEPASISCRSSQSLVHSNGNTYLQ |
| | | WYLQKPGQSPQLLIYKVSNRFSGVPDRFSGSGSGTDFTLKISRVEAEDVGVYYCSQSI |
| | | YVPYTFGQGTKLEIKR |
| 11 | Amino acid | MALLLALSLLVLWTSPAPTLSGTNDAEDCCLSVTQKPIPGYIVRNFHYLLIKDGCR |
| | sequence of | VPAVVFTTLRGRQLCAPPDQPWVERIIQRLQRTSAKMKRRSS |
| | human CCL19 | |
| 12 | Nucleic acid | Atggccccccgtgtgaccccactcctggccttcagcctgctggttctctggaccttcccagccccaactctgggggg |
| | sequence of | tgctaatgAtgcggaagactgctgcctgtctgtgacccagcgccccatccctgggaacatcgtgaaagccttccgct |
| | murine CCL19 | accttcttaatgaagatGgctgcagggtgcctgctgttgtgttcaccacactaaggggctatcagctctgtgcacct |
| | | ccagaccagccctgggtggatcgcatcatccgaagactgaagaagtcttctgccaagaacaaaggcaacagcaccag |
| | | aaggagccctgtgtct |
| 13 | Amino acid | EVQLVQSGAEVKKPGASVKVSCKASGYTFSDYEMHWVRQAPGQGLEWMGAIHP |
| | sequence of | GSGDTAYNQRFKGRVTITADKSTSTAYMELSSLRSEDTAVYYCARFYSYAYWGQ |
| | GPC3-28ZCAR | GTLVTVSAGGGGSGGGGSGGGGSDIVMTQTPLSLPVTPGEPASISCRSSQSLVHSN |
| | | GNTYLQWYLQKPGQSPQLLIYKVSNRFSGVPDRFSGSGSGTDFTLKISRVEAEDVG |
| | | VYYCSQSIYVPYTFGQGTKLEIKRTTTPAPRPPTPAPTIASQPLSLRPEACRPAAGGA |
| | | VHTRGLDFACDFWVLVVVGGVLACYSLLVTVAFIIFWVRSKRSRLLHSDYMNMT |
| | | PRRPGPTRKHYQPYAPPRDFAAYRSRVKFSRSADAPAYQQGQNQLYNELNLGRR |
| | | EEYDVLDKRRGRDPEMGGKPQRRKNPQEGLYNELQKDKMAEAYSEIGMKGERR |
| | | RGKGHDGLYQGLSTATKDTYDALHMQALPPR |
| 14 | Amino acid | EVQLVQSGAEVKKPGASVKVSCKASGYTFSDYEMHWVRQAPGQGLEWMGAIHPG |
| | sequence of | SGDTAYNQRFKGRVTITADKSTSTAYMELSSLRSEDTAVYYCARFYSYAYWGQGT |
| | GPC3-BBZCAR | LVTVSAGGGGSGGGGSGGGGSDIVMTQTPLSLPVTPGEPASISCRSSQSLVHSNGN |
| | | TYLQWYLQKPGQSPQLLIYKVSNRFSGVPDRFSGSGSGTDFTLKISRVEAEDVGVY |
| | | YCSQSIYVPYTFGQGTKLEIKRTTTPAPRPPTPAPTIASQPLSLRPEACRPAAGGAVH |
| | | TRGLDFACDIYIWAPLAGTCGVLLLSLVITLYCKRGRKKLLYIFKQPFMRPVQTTQE |
| | | EDGCSCRFPEEEEGGCELRVKFSRSADAPAYQQGQNQLYNELNLGRREEYDVLDK |
| | | RRGRDPEMGGKPQRRKNPQEGLYNELQKDKMAEAYSEIGMKGERRRGKGHDGL |
| | | YQGLSTATKDTYDALHMQALPPR |
| 15 | Amino acid | EVQLVQSGAEVKKPGASVKVSCKASGYTFSDYEMHWVRQAPGQGLEWMGAIHP |
| | sequence of | GSGDTAYNQRFKGRVTITADKSTSTAYMELSSLRSEDTAVYYCARFYSYAYWGQ |
| | GPC3-28BBZ | GTLVTVSAGGGGSGGGGSGGGGSDIVMTQTPLSLPVTPGEPASISCRSSQSLVHSN |
| | | GNTYLQWYLQKPGQSPQLLIYKVSNRFSGVPDRFSGSGSGTDFTLKISRVEAEDV |
| | | GVYYCSQSIYVPYTFGQGTKLEIKRTTTPAPRPPTPAPTIASQPLSLRPEACRPAAG |
| | | GAVHTRGLDFACDFWVLVVVGGVLACYSLLVTVAFIIFWVRSKRSRLLHSDYM |
| | | NMTPRRPGPTRKHYQPYAPPRDFAAYRSKRGRKKLLYIFKQPFMRPVQTTQEED |
| | | GCSCRFPEEEEGGCELRVKFSRSADAPAYQQGQNQLYNELNLGRREEYDVLDK |
| | | RRGRDPEMGGKPQRRKNPQEGLYNELQKDKMAEAYSEIGMKGERRRGKGHD |
| | | GLYQGLSTATKDTYDALHMQALPPR |
| 16 | Nucleic acid | Ggaggaaaaactgtttcatacagaaggcgtggaggaaaaactgtttcatacagaaggcgtggaggaaaaact |
| | sequence of | Gtttcatacagaaggcgtcaattgtcctcgacggaggaaaaactgtttcatacagaaggcgtggaggaaaa |
| | NFAT6 binding | actgtttcatacagaaggcgtggaggaaaaactgtttcatacagaaggcgt |
| | motif | |
| 17 | Nucleic acid | atctacatctgggcgcccttggccgggacttgtggggtccttctcctgtcactggttatcaccCTTTACTGC |
| | sequence of | |
| | the | |
| | transmembrane | |
| | domain of CD8 | |
| 18 | Amino acid | Krgrkkllyifkqpfmrpvqttqeedgcscrfpeeeeggcel |
| | sequence of | |
| | the | |
| | intracellular | |
| | domain of | |
| | CD137 | |
| 19 | Nucleic acid | Agagtgaagttcagcaggagcgcagacgcccccgcgtaccagcagggccagaaccagctctataacgagctcaatct |
| | sequence of | aggacGaagagaggagtacgatgttttggacaagagacgtggccgggaccctgagatggggggaaagccgcagagaa |
| | the | ggaagaaccCtcaggaaggcctgtacaatgaactgcagaaagataagatggcggaggcctacagtgagattgggatg |
| | intracellular | aaaggcgagcgccggAggggcaaggggcacgatggcctttaccagggtctcagtacagccaccaaggacacctacga |
| | domain of CD3ζ | cgcccttcacatgcaggccctgccccctcgc |
| 20 | Nucleic acid | atggccttaccagtgaccgccttgctcctgccgctggccttgctgctccacgccgccaggccg |
| | sequence of | |
| | CD8α signal | |
| | peptide | |
| 21 | Nucleic acid | Accacgacgccagcgccgcgaccaccaacaccggcgcccaccatcgcgtcgcagcccctgtccctgcgcccagaggc |
| | sequence of | gtgccggccagcggcggggggcgcagtgcacacgagggggctggacttcgcctgtgat |
| | the hinge | |
| | region of CD8α | |
| 22 | NFAT binding | ggaggaaaaactgtttcatacagaaggcgt |
| | motif | |
| 23 | Amino acid | Qvqlqesgpglikpsqtlsltctvsggsissgynwhwirqppgkglewigyihytgstnynpalrsrvtisvdtsk |
| | sequence of | Nqfslklssvtaadtaiyycariyngnsfpywgqgttvtvssggggsggggsggggsdivmtqspdslavslgera |
| | claudin18.2 | Tinckssqslfnsgnqknyltwyqqkpgqppklliywastresgvpdrfsgsgsgtdftltisslqaedvavyyc |
| | scFV | qnaysfpytfgggtkleikr |
| 24 | Amino acid | Qvqlqesgpglikpsqtlsltctvsggsissgynwhwirqppgkglewigyihytgstnynpalrsrvtisvdtskn |
| | sequence of | Qfslklssvtaadtaiyycariyngnsfpywgqgttvtvssggggsggggsggggsdivmtqspdslavslgerati |
| | claudin18.2-- | Nckssqslfnsgnqknyltwyqqkpgqppklliywastresgvpdrfsgsgsgtdftltisslqaedvavyycqnay |
| | 28 | sfpytfgggtkleikrTttpaprpptpaptiasqplslrpeacrpaaggavhtrgldfacdFwvlvvvggvlacysl |
| | ZCAR | lvtvafiifwvRskrsrllhsdymnmtprrpgptrkhyqpyapprdfaayrsrvkfsrsadapayqqgqnqlynel |
| | | nlgrreeydvldkrrgrdpemggkpqrrknpqeglynelqkdkmaeayseigmkgerrrgkghdglyqglstatkd |
| | | tydalhmqalppr |
| 25 | Amino acid | QVQLQESGPGLIKPSQTLSLTCTVSGGSISSGYNWHWIRQPPGKGLEWIGYIHYTGSTNYNP |
| | sequence of | ALRSRVTISVDTSKNQFSLKLSSVTAADTAIYYCARIYNGNSFPYWGQGTTVTVSSGGGGS |
| | claudin18.2-- | GGGGSGGGGSDIVMTQSPDSLAVSLGERATINCKSSQSLFNSGNQKNYLTWYQQKPGQPP |
| | B | KLLIYWASTRESGVPDRFSGSGSGTDFTLTISSLQAEDVAVYYCQNAYSFPYTFGGGTKLEI |
| | BZCAR | KRTTTPAPRPPTPAPTIASQPLSLRPEACRPAAGGAVHTRGLDFACDIYIWAPLAGTCGVLL |
| | | LSLVITLYCKRGRKKLLYIFKQPFMRPVQTTQEEDGCSCRFPEEEEGGCELRVKFSRSADAP |
| | | AYQQGQNQLYNELNLGRREEYDVLDKRRGRDPEMGGKPQRRKNPQEGLYNELQKDKMA |
| | | EAYSEIGMKGERRRGKGHDGLYQGLSTATKDTYDALHMQALPPR |
| 26 | Amino acid | Qvqlqesgpglikpsqtlsltctvsggsissgynwhwirqppgkglewigyihytgstnynpalrsrvtisv |
| | sequence of | Dtsknqfslklssvtaadtaiyycariyngnsfpywgqgttvtvssggggsggggsggggsdivmtqspdsl |
| | claudin18.2- | Avslgeratinckssqslfnsgnqknyltwyqqkpgqppklliywastresgvpdrfsgsgsgtdftltiss |
| | 28 | lqaedvavyycqnaysfpytfgggtkleikrTttpaprpptpaptiasqplslrpeacrpaaggavhtrgld |
| | BBZ CAR | facdFwvlvvvggvlacysllvtvafiifwvRskrsrllhsdymnmtprrpgptrkhyqpyapprdfaayrs |
| | | Krgrkkllyifkqpfmrpvqttqeedgcscrfpeeeeggcelrvkfsrsadapayqqgqnqlynelnlgrre |
| | | Eydvldkrrgrdpemggkpqrrknpqeglynelqkdkmaeayseigmkgerrrgkghdglyqglstatkdty |
| | | dalhmqalppr |
| 27 | Nucleotide | Gaggtgcagctggtgcagagcggcgccgaggtgaagaagcccggcgccagcgtgaaggtgagctgcaagg |
| | sequence of | Ccagcggctacaccttcagcgactacgagatgcactgggtgcggcaggcccccggccagggcctggagtg |
| | GPC3 scFV | Gatgggcgccatccaccccggcagcggcgacaccgcctacaaccagcggttcaagggccgggtgaccatc |
| | | Accgccgacaagagcaccagcaccgcctacatggagctgagcagcctgcggagcgaggacaccgccgtgt |
| | | Actactgcgcccggttctacagctacgcctactggggccagggcaccctggtgaccgtgagcgccggtgg |
| | | Aggcggttcaggcggaggtggttctggcggtggcggatcggacatcgtgatgacccagacccccctgagc |
| | | Ctgcccgtgacccccggcgagcccgccagcatcagctgccggagcagccagagcctggtgcacagcaacg |
| | | Gcaacacctacctgcagtggtacctgcagaagcccggccagagcccccagctgctgatctacaaggtgag |
| | | Caaccggttcagcggcgtgcccgaccggttcagcggcagcggcagcggcaccgacttcaccctgaagatc |
| | | Agccgggtggaggccgaggacgtgggcgtgtactactgcagccagagcatctacgtgccctacaccttcg |
| | | gccagggcaccaagctggagatcaaacgt |
| 28 | Nucleotide | Atgttccatgtttcttttaggtatatctttggacttcctcccctgatccttgttctgttgccagtagcatcatctga |
| | sequence of | ttgtgatattgaaggtaaagatggcaaacaatatgagagtgttctaatggtcagcatcgatcaattattggacagca |
| | Human IL7 | tgaaagaaattggtagcaattgcctgaataatgaatttaacttttttaaaagacatatctgtgatgctaataaggaa |
| | | ggtatgtttttattccgtgctgctcgcaagttgaggcaatttcttaaaatgaatagcactggtgattttgatctcca |
| | | cttattaaaagtttcagaaggcacaacaatactgttgaactgcactggccaggttaaaggaagaaaaccagctgccc |
| | | tgggtgaagcccaaccaacaaagagtttggaagaaaataaatctttaaaggaacagaaaaaactgaatgacttgtgt |
| | | ttcctaaagagactattacaagagataaaaacttgttggaataaaattttgatgggcactaaagaacac |
| 29 | Nucleotide | atggctcagtcactggctctgagcctccttatcctggttctggcctttggcatccccaggacccaaggcagtgatgg |
| | sequence of | aggggctcaggactgttgcctcaagtacagccaaaggaagattcccgccaaggttgtccgcagctaccggaagcagg |
| | human CCL21 | aaccaagcttaggctgctccatcccagctatcctgttcttgccccgcaagcgctctcaggcagagctatgtgcagac |
| | | ccaaaggagctctgggtgcagcagctgatgcagcatctggacaagacaccatccccacagaaaccagcccagggctg |
| | | caggaaggacaggggggcctccaagactggcaagaaaggaaagggctccaaaggctgcaagaggactgagcggtcac |
| | | agacccctaaagggcca |
| 30 | Nucleotide | atggccctgctactggccctcagcctgctggttctctggacttccccagccccaactctgagtggcaccaatgatgc |
| | sequence of | tgaagactgctgcctgtctgtgacccagaaacccatccctgggtacatcgtgaggaacttccactaccttctcatca |
| | human CCL19 | aggatggctgcagggtgcctgctgtagtgttcaccacactgaggggccgccagctctgtgcacccccagaccagccc |
| | | tgggtagaacgcatcatccagagactgcagaggacctcagccaagatgaagcgccgcagcagt |
| 31 | Amino acid | mfhvsfryifgipplilvllpvtssechikdkegkayesvlmisideldkmtgtdsncpnnepnffrkhvcddtkea |
| | sequence of | aflmaarklkqflkmniseefnvhlltvsqgtqtlvnctskeeknvkeqkkndacflkrllreiktcwnkilkgsi |
| | murine IL7 | |
| 32 | Amino acid | maqmmtlsllslvlalcipwtqgsdgggqdcclkysqkkipysivrgyrkqepslgcpipailfsprkhskpelcan |
| | sequence of | peegwvqnlmrrldqppapgkqspgcrknrgtsksgkkgkgskgckrteqtqpsrg |
| | murine CCL21a | |
| 33 | Amino acid | maqmmtlsllslvlalcipwtqgsdgggqdcclkysqkkipysivrgyrkqepslgcpipailflprkhskpelcan |
| | sequence of | peegwvqnlmrrldqppapgkqspgcrknrgtsksgkkgkgskgckrteqtqpsrg |
| | murine CCL21b | |
| 34 | Amino acid | maprvtpllafsllvlwtfpaptlggandaedcclsvtqrpipgnivkafryllnedgcrvpavvfttlrgyqlcap |
| | sequence of | pdqpwvdriirrlkkssaknkgnstrrspvs |
| | murine CCL19 | |
| 35 | Amino acid | MAGTVRTACLVVAMLLSLDFPGQAQPPPPPPDATCHQVRSFFQRLQPGLKWVPETPVPGS |
| | sequence of | DLQVCLPKGPTCCSRKMEEKYQLTARLNMEQLLQSASMELKFLIIQNAAVFQEAFEIVVRH |
| | human GPC3 | AKNYTNAMFKNNYPSLTPQAFEFVGEFFTDVSLYILGSDINVDDMVNELFDSLFPVIYTQL |
| | | MNPGLPDSALDINECLRGARRDLKVFGNFPKLIMTQVSKSLQVTRIFLQALNLGIEVINTTD |
| | | HLKFSKDCGRMLTRMWYCSYCQGLMMVKPCGGYCNVVMQGCMAGVVEIDKYWREYIL |
| | | SLEELVNGMYRIYDMENVLLGLFSTIHDSIQYVQKNAGKLTTTIGKLCAHSQQRQYRSAYY |
| | | PEDLFIDKKVLKVAHVEHEETLSSRRRELIQKLKSFISFYSALPGYICSHSPVAENDTLCWNG |
| | | QELVERYSQKAARNGMKNQFNLHELKMKGPEPVVSQIIDKLKHINQLLRTMSMPKGRVLD |
| | | KNLDEEGFESGDCGDDEDECIGGSGDGMIKVKNQLRFLAELAYDLDVDDAPGNSQQATPK |
| | | DNEISTFHNLGNVHSPLKLLTSMAISVVCFFFLVH |
| 36 | Intracellular | Aaacggggcagaaagaaactcctgtatatattcaaacaaccatttatgagaccagtacaaactactcaagaggaaga |
| | signaling | tggctgtagctgccgatttccagaagaagaagaaggaggatgtgaactg |
| | domain of | |
| | CD137 | |

All documents mentioned herein are incorporated by reference in this application as if each document are individually incorporated by reference. In addition, it should be understood that after reading the above teaching content of the present invention, those skilled in the art may make various changes or modifications to the present invention, and these equivalent forms also fall within the scope defined by the appended claims of the present application.

1-40. (canceled)

41. A genetically engineered immune effector cell, characterized in that the immune effector cell expresses a receptor specifically recognizing a target antigen and IL7, and the IL-7 is inducibly expressed and regulated by the receptor.

42. The immune effector cell according to claim 41, characterized in that the expression of IL-7 may be initiated when the receptor recognizes the target antigen; preferably, the receptor induces the expression of the IL-7 through an inducible promoter.

43. The immune effector cell according to claim 41, characterized in that the immune effector cell further expresses a chemokine, a chemokine receptor, a cytokine other than IL-7, siRNA reducing PD-1 expression, a protein blocking the binding of PD-L1 to PD-1, or a safety switch.

44. The immune effector cell according to claim 43, characterized in that the chemokine is a lymphocyte chemokine; preferably, the lymphocyte chemokine is CCL21 or CCL19; or the chemokine receptor is selected from the group consisting of: CCR2, CCR5, CXCR2, and CXCR4.

45. The immune effector cell according to claim 43, characterized in that the other cytokine is selected from the group consisting of: IL-15, IL-21, IL18, and type I interferon.

46. The immune effector cell according to claim 43, characterized in that the protein blocking the binding of PD-L1 to PD-1 is selected from the group consisting of: a PD-L1 antibody, a PD-1 antibody, a natural PD-1 or a truncated fragment of the natural PD-1, and a fusion peptide containing the natural PD-1 or the truncated fragment of the natural PD-1.

47. The immune effector cell according to claim 43, characterized in that the safety switch is selected from the group consisting of: iCaspase-9, truancated EGFR, RQR8, and a protein with a killing effect on immune effector cells.

48. The immune effector cell according to claim 41, characterized in that the immune effector cell is selected from the group consisting of: a T cell, an NK cell, an NKT cell, a mast cell, a macrophage, a dendritic cell, a CIK cell, and a stem cell-derived immune effector cell;
preferably, the immune effector cell is a T cell.

49. The immune effector cell according to claim 42, characterized in that the inducible promoter comprises a binding motif of a transcription factor, and the activation of the inducible promoter is dependent on the activation of the receptor or is dependent on the binding of the receptor to the target antigen; preferably, the binding motif comprises an NFAT, NF-κB or AP-1 binding motif, or a combination of at least two of NFAT, NF-κB and AP-1 binding motifs;
more preferably, the binding motif is an NFAT binding motif; still more preferably, the sequence of the NFAT binding motif is represented by SEQ ID NO: 22.

50. The immune effector cell according to claim 49, characterized in that the binding motif comprises 1-12 NFAT binding motifs, 1-12 NF-κB binding motifs, 1-12 AP-1 binding motifs, or a combination of at least two of 1-12 NFAT, 1-12 NF-κB and 1-12 AP-1 binding motifs; preferably, the binding motif comprises 1-6 NFAT binding motifs, 1-6 NF-κB binding motifs, 1-6 AP-1 binding motifs, or a combination of at least two of 1-6 NFAT, 1-6 NF-κB and 1-6 AP-1 binding motifs.

51. The immune effector cell according to claim 49, characterized in that the inducible promoter of the immune cell further comprises a minimal promoter operably linked to the binding motif; preferably, the minimal promoter is a cytokine minimal promoter; more preferably, the minimal promoter includes interleukin, interferon, tumor necrosis factor superfamily, colony stimulating factor, chemokine, and growth factor minimal promoter; further preferably, the minimal promoter is an IFN-γ, a TNF-α, or an IL-2 minimal promoter; more preferably, the minimal promoter is an IL-2 minimal promoter; still more preferably, the sequence of the IL-2 minimal promoter is represented by SEQ ID NO: 3.

52. The cell according to claim 41, characterized in that the IL-7 is natural IL-7, or a truncated fragment of natural IL-7 or a mutant of natural IL-7 having the same function as natural IL-7; preferably, the natural IL-7 has at least 90% identity with the amino acid sequence represented by SEQ ID NO: 1 or SEQ ID NO: 31, or is a truncated fragment of the amino acid sequence represented by SEQ ID NO: 1 or SEQ ID NO: 31; alternatively, has at least 90% identity with the amino acid sequence encoded by the nucleotide represented by SEQ ID NO: 2 or SEQ ID NO: 28, or is a truncated fragment of the amino acid sequence encoded by the nucleotide represented by SEQ ID NO: 2 or SEQ ID NO: 28.

53. The cell according to claim 44, characterized in that the CCL21 is natural CCL21, or a truncated fragment of natural CCL21 or a mutant of natural CCL21 having the same function as natural CCL21; preferably, the natural CCL21 has at least 90% identity with the amino acid sequence represented by SEQ ID NO: 7, SEQ ID NO: 32 or SEQ ID NO: 33, or is a truncated fragment of the amino acid sequence represented by SEQ ID NO: 7, SEQ ID NO: 32 or SEQ ID NO: 33; alternatively, has at least 90% identity with the amino acid sequence encoded by the nucleotide represented by SEQ ID NO: 8, SEQ ID NO: 9 or SEQ ID NO: 29, or is a truncated fragment of the amino acid sequence encoded by the nucleotide represented by SEQ ID NO: 8, SEQ ID NO: 9 or SEQ ID NO: 29;
alternatively, the CCL19 is natural CCL19, or a truncated fragment of natural CCL19 or a mutant of natural CCL19 having the same function as natural CCL19; preferably, the natural CCL19 has at least 90% identity with the amino acid sequence represented by SEQ ID NO: 11 or SEQ ID NO: 34, or is a truncated fragment of the amino acid sequence represented by SEQ ID NO: 11 or SEQ ID NO: 34; alternatively, has at least 90% identity with the amino acid sequence encoded by the nucleotide represented by SEQ ID NO: 12 or SEQ ID NO: 30, or is a truncated fragment of the amino acid sequence encoded by the nucleotide represented by SEQ ID NO: 12 or SEQ ID NO: 30.

54. The cell according to claim 43, characterized in that the chemokine, the chemokine receptor, the cytokine other than IL-7, the siRNA reducing PD-1 expression, the protein blocking the binding of PD-L1 to PD-1, or the safety switch is either constitutively expressed or inducibly expressed.

55. The cell according to claim 41, characterized in that the target antigen is a tumor antigen and/or a pathogen antigen; preferably, a tumor antigen; more preferably, the target antigen is a solid tumor antigen; still more preferably, the solid tumor antigen is GPC3, EGFR, EGFRvIII, mesothelin or Claudin18.2.

56. The cell according to claim 55, characterized in that the receptor is selected from the group consisting of: a chimeric antigen receptor (CAR), a T cell receptor (TCR), a T cell fusion protein (TFP), a T cell antigen coupler (TAC), and a combination thereof; preferably, the receptor is a chimeric antigen receptor; more preferably, the chimeric antigen receptor comprises:
(i) an antibody or fragment thereof specifically binding the target antigen, a transmembrane domain of CD28 or CD8, a costimulatory signaling domain of CD28, and an intracellular signaling domain of CD3ζ; or
(ii) an antibody or fragment thereof specifically binding the target antigen, a transmembrane domain of CD28 or CD8, a costimulatory signaling domain of 4-1BB, and an intracellular signaling domain of CD3ζ; or
(iii) an antibody or fragment thereof specifically binding the target antigen, a transmembrane domain of CD28 or CD8, a costimulatory signaling domain of CD28, a costimulatory signaling domain of 4-1BB, and an intracellular signaling domain of CD3ζ.

57. The cell according to claim 56, characterized in that the amino acid sequence of the antigen binding domain of the chimeric antigen receptor (CAR), T cell fusion protein (TFP), or T cell antigen coupler (TAC) has at least 90% identity with the amino acid sequence represented by SEQ ID NO: 10 or SEQ ID NO: 23; preferably, the amino acid sequence of the receptor has at least 90% identity with the amino acid sequence represented by SEQ ID NO: 13, 14, 15, 24, 25, or 26.

58. The cell according to claim 51, characterized in that the nucleic acid sequence of the immune cell-inducible promoter inducing the expression of IL7 is represented by SEQ ID NO: 4.

59. The cell according to claim 41, characterized in that the receptor and IL-7 are expressed by the same nucleic acid molecule, or expressed by different nucleic acid molecules;
preferably, the receptor and IL-7 are expressed by the same nucleic acid molecule; an expression cassette of the IL-7 and the receptor, and an expression cassette and another expression cassette are directly connected or connected by a tandem fragment, wherein the tandem fragment is selected from the group consisting of: F2A, PA2, T2A, and/or E2A.

60. A nucleic acid molecule, which expresses the IL-7 according to claim 41, or IL-7 and a chemokine, a chemokine receptor, a cytokine other than IL-7, a siRNA reducing PD-1 expression, a protein blocking the binding of PD-L1 to PD-1, or a safety switch; and the nucleic acid molecule also expresses the receptor specifically recognizing the target antigen according to claim 41; preferably the nucleic acid consists of DNA and/or RNA; alternatively, the nucleic acid is mRNA, or the nucleic acid comprises a nucleotide analog.

61. A method for treating a mammal suffering from a disease related to the expression of GPC3 or claudin18.2, comprising administering an effective amount of the cells according to claim 41 to the mammal; preferably, the disease related to the expression of GPC3 or claudin18.2 is selected from the group consisting of: colon cancer, rectal cancer, renal cell cancer, liver cancer, lung cancer, small intestine cancer, esophagus cancer, melanoma, bone cancer, pancreatic cancer, skin cancer, head and neck cancer, skin or intraocular malignant melanoma, uterine cancer, ovarian cancer, rectal cancer, anal cancer, stomach cancer, testicular cancer, uterine cancer, fallopian tube cancer, endometrial cancer, cervical cancer, vaginal cancer, endocrine system cancer, thyroid cancer, parathyroid cancer, adrenal cancer, soft tissue sarcoma, urethral cancer, penile cancer, bladder cancer, kidney or ureter cancer, renal pelvis cancer, central nervous system (CNS) tumor, tumor angiogenesis, spinal tumor, brain stem glial tumor, pituitary adenoma, Kaposi's sarcoma, epidermoid carcinoma, squamous cell carcinoma, non-cancer related indications related to GPC3 or claudin18.2 expression; preferably, selected from the group consisting of: liver cancer, lung cancer, breast cancer, ovarian cancer, kidney cancer, thyroid cancer, stomach cancer, colorectal cancer, pancreatic cancer, and esophageal cancer; more preferably, the cells according to claim 41 are administered in combination with an agent increasing the efficacy of the cells according to claim 41; preferably, in combination with a chemotherapeutic agent; more preferably, the cells according to claim 41 are administered in combination with an agent ameliorating one or more side effects associated with administration of the cells according to claim 41; more preferably, the cells according to claim 41 are administered in combination with an agent for treating a disease associated with GPC3 or claudin 18.2, preferably in combination with a chemotherapeutic agent.
